(19) United States
(12) Patent Application Publication
(10) Pub. No.: US 2008/0113900 A1
(43) Pub. Date: May 15, 2008

(54) Pharmaceutical compositions and therapeutic applications for the use of a synthetic vitamin B12 derivative, glutathionylcobalamin

(76) Inventors: Nicola E. Brasch, Kent, OH (US); Catherine Stephanie Birch, Cheshire (GB); John Henry Howatson Williams, Corwen (GB)

Correspondence Address: HUDAK, SHUNK & FARINE, CO., L.P.A., 2020 FRONT STREET, SUITE 307, CUYAHOGA FALLS, OH, 44221, US

(21) Appl. No.: 11/901,983

(22) Filed: Sep. 20, 2007

Related U.S. Application Data
(60) Provisional application No. 60/846,435, filed on Sep. 22, 2006.

(51) Int. Cl. A61K 38/06; A61P 25/00; A61P 25/28; A61P 27/06; A61P 9/00

(52) U.S. Cl. 514/6

(57) ABSTRACT

Therapeutic applications, such as prevention, treatment and supplementation, for the use of novel and other thiolatocobalamins to protect human cells against the effects of oxidative stress. In particular, this invention relates to the use of a novel synthetic thiolatocobalamin, glutathionylcobalamin to protect animal cells against oxidative stress damage. This invention also relates to the use of thiolatocobalamins, such as glutathionylcobalamin, in lieu of current, commercially available forms of vitamin B₁₂ for the treatment and prevention of conditions associated with oxidative stress damage and for dietary supplementation.

### CROSS-REFERENCE

This application claims priority to U.S. Provisional Application Ser. No. 60/846,435 filed on Sep. 22, 2006.

### FIELD OF THE INVENTION

This invention relates to compositions having therapeutic applications, such as prevention, treatment and supplementation, for the use in protecting animal, and in particular, human cells against the effects of oxidative stress. This invention relates to a novel synthetic thiolatocobalamin, glutathionylcobalamin, which can be used to protect human cells against oxidative stress damage. This invention also relates to the use of thiolatocobalamins as a pharmaceutical composition and as a dietary supplement, such as glutathionylcobalamin, in lieu of current, commercially available forms of vitamin B₁₂, for the treatment and prevention of conditions associated with oxidative stress damage.

### BACKGROUND OF THE INVENTION

Three forms of vitamin B₁₂ have long been recognized to occur in biology, aquacobalamin/hydroxycobalamin, methylcobalamin (MeCbl) and adenosylcobalamin (AdoCbl) (Golding, B. T. Chem. Brit. 1990, 950). (See Formula I). Methylcobalamin (MeCbl) and adenosylcobalamin (AdoCbl) play crucial roles in the B₁₂-dependent enzyme reactions and are frequently referred to as the B₁₂ co-enzymes. Two known B₁₂-dependent enzymes exist in humans: methionine synthase, which is methylcobalamin (MeCbl)-dependent, and methylmalonyl-coenzyme A mutase, which is adenosylcobalamin (AdoCbl)-dependent. (Dolphin, D. (ed). B₁₂; John Wiley & Sons, Inc.: New York, USA, 1982; Banerjee, R. (ed.) Chemistry and Biochemistry of B₁₂; JohnWiley & Sons, Inc.: New York, USA, 1999). In short, methionine synthase and methylmalonyl-CoA mutase require the vitamin B₁₂ derivatives methylcobalamin (MeCbl) and adenosylcobalamin (AdoCbl), respectively, for certain enzymatic reactions in the body. For example, in the MeCbl-dependent methionine synthase reaction, a methyl group is transferred from methyl-tetrahydrofolate (a metabolite of folate) to homocysteine (Hcy) via MeCbl to give methionine and tetrahydrofolate. This reaction results in the conversion of homocysteine, an amino acid found in humans, which has destructive, oxidative properties, back to methionine. This reaction has received much attention in the medical literature in recent years, because its impairment can lead to elevated levels of homocysteine, which is associated with an increased risk of cardiovascular, cerebrovascular and peripheral vascular disease, and other pathological conditions which are discussed below.

Thiol derivatives of B₁₂, thiolatocobalamins, were first identified in the 1960's, but have not attracted much attention until recently. They are characterized by having a cobalt-sulphur bond in the upper (beta) axial position. (See Formula 1). Glutathionylcobalamin (GSCbl (or GluSCbl), a thiolatocobalamin) has been recently isolated from mammalian cells. A method for preparing glutathionylcobalamin is the subject of U.S. Pat. No. 7,030,105, the contents of which are incorporated herein by reference.

Glutathionylcobalamin (GSCbl) is an important cobalamin metabolite in mammals and is more active than other cobalamins in promoting methionine synthase activity in rabbit spleen extracts. It has been proposed that, in vivo, GSCbl (or a closely related thiolatocobalamin adduct) is a precursor of the two coenzyme forms of vitamin B₁₂—AdoCbl and MeCbl. An alternative role for GSCbl was also recently proposed, in which the formation of GSCbl prevents B₁₂ from being scavenged by xenobiotics.

The exact biochemical pathway(s) that lead to the incorporation of cobalamins into the B₁₂-dependent enzymes have not yet been elucidated. It is known that thiolatocobalamins can be reduced by free thiols, yielding cob(l)alamin species, which can in turn be methylated by S-adenosylmethionine to form methylcobalamin. Whether this is an important biochemical pathway in humans needs further study.

A variety of thiolatocobalamins have been synthesized. Recently, simple synthetic methods have been reported for the preparation of three additional thiolatocobalamins—D,L-homocysteinyl-cobalamin (HcyCbl), the sodium salt of N-acetyl-L-cysteinylcobalamin (Na[NACCbl]), and 2-N-acetylamino-2-carbomethoxy-L-ethanethiolatocobalamin (NACMECbl). (Suarez-Moreira, E., Hannibal, L., Smith, C., Chavez, R., Jacobsen, D. W., and Brasch, N. E., Dalton Trans., in press). However GSCbl is the only thiolatocobalamin that has been isolated in mammals to date. Furthermore, prior to this synthesis, Na[NACCbl] and NACMECbl were not even reported to exist.

Formula I, below, depicts the structures of vitamin B₁₂, including the two coenzyme forms of vitamin B₁₂ and related B₁₂ derivatives found in humans, all commonly referred to as the cobalamins.

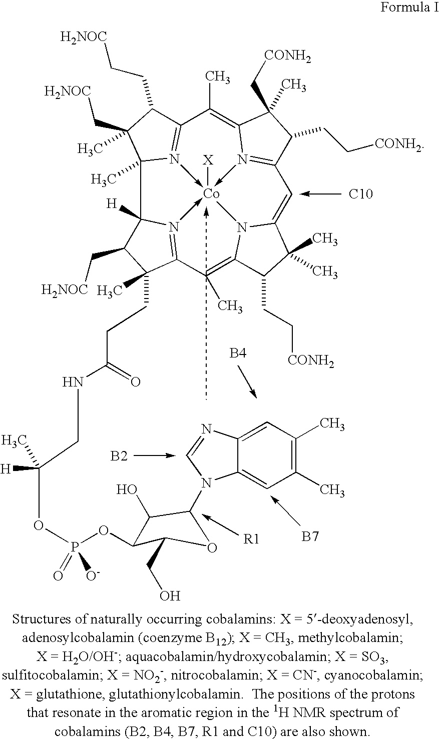

The cobalamins belong to a family of compounds known as the corrinoids, which differ from one another in the specific nucleotide occupying the a axial site of the cobalt-corrin complex. The α (or lower) axial site is occupied by an intramolecularly-bound 5,6-dimethylbenzimidazole, and the β (or upper) axial site can be occupied by a variety of ligands. The various thiol ligand structures for the thiolatocobalamins mentioned herein are shown below.

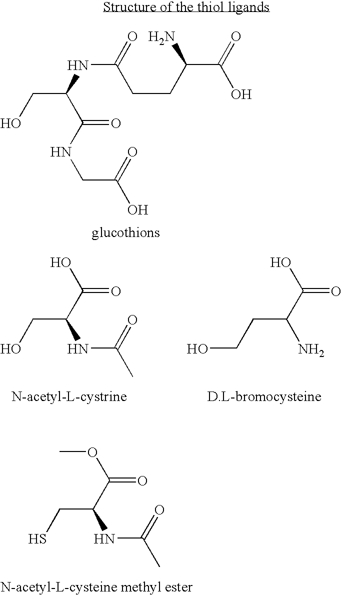

It is known that vitamin B₁₂ and its derivatives play key roles in human, animal and microbial metabolism. In humans, vitamin B₁₂ helps maintain healthy nerve cells and red blood cells. It is also needed to produce DNA, the genetic material in all cells. (National Institutes of Health, Office of Dietary Supplements, Dietary Fact Sheet: Vitamin B₁₂). Cobalamins (Cbls) are bound to protein in food, and hydrochloric acid in the stomach releases vitamin B₁₂ from proteins during digestion. Once released, Cbls combine with a protein known as salivary haptocorrin (HC, also known as R-binder). Upon pancreatic proteolytic degradation of HC, Cbl is transferred in the duodenum to intrinsic factor (IF), which can then be absorbed by the GI tract. Cbl is transferred to transcobalamin (TC, TCII) within enterocytes. A substantial portion of TC-Cbl entering the portal vein after absorption is cleared by hepatocytes. Any free Cbl entering the circulation binds to either TC or HC.

Vitamin B₁₂ deficiencies can occur in humans in a number of circumstances. Deficiencies can occur from malabsorption problems (damage to the GI tract lining, achlorhydria, inflammatory bowel conditions, infections, lack of intrinsic factor or other genetic anomalies), lack of a diet rich in vitamin B₁₂, or the inability to utilize absorbed vitamin B₁₂ and enzymatic or amino acid deficiencies. Certain drugs can also interfere with the absorption of vitamin B₁₂.

Vitamin B₁₂ deficiency can manifest in several different ways, including but not limited to anemias (including megaloblastic anemia also known as pernicious anemia), weakness, fatigue, weight loss, neurological changes, such as neuropathies (numbness and tingling), depression, confusion, and cognitive decline (such as loss of memory and dementia).

Vitamin B₁₂, along with folate and vitamin B₆, are involved in homocysteine metabolism. Homocysteine is a non-protein amino acid reversibly formed and secreted during human metabolism. Homocysteine is, however, a neurotoxin, and an abnormal increase in plasma homocysteine levels has been implicated in many pathological conditions, such as cardiovascular disease, neural tube defects, osteoporosis, stroke and other cerebrovascular disease, peripheral vascular disease, and certain forms of glaucoma and is now recognized in Alzheimer's disease. (Tchantchou, F., “Homocysteine metabolism and various consequences of folate deficiency”. J.Alzheimees Dis. August 2006; Vol. 9, No. 4: 421-27). Homocysteine is eliminated from the body and is regulated by the transmethylation and transsulfuration pathways.

Homocysteine, among other reactive species, plays a key role in inducing oxidative stress. Oxidative stress can be defined as a harmful condition that occurs when there is an excess of free radicals, a decrease in antioxidants, or both. (E.g., Halliwell B. Introduction: Free Radicals and Human Disease—Trick or Treat? In: Thomas, C. E., Kalyanaraman, B. (ed.) Oxygen Radicals and the Disease Process. 1^{st} ed. Amsterdam. Harwood Academic Publishers. 1997. pp. 1-14). Free radicals cause damage to cells by attacking their lipids, proteins and DNA components. A free radical is any species that contains one or more unpaired electrons, which makes it more reactive so that it can react with other species to form new free radicals. (Goodall, H. Oxidative stress: an overview.) It is this cycle that can lead to damage to cells in the body from prolonged exposure to free radicals.

The term reactive species is used to describe free radicals and other molecules that are themselves easily converted to free radicals or are powerful oxidizing agents. (Id.) Hydrogen peroxide is another example of a reactive species found intracellularly and extracellularly in humans.

It is known that a deficiency of vitamin B₁₂, folate, or vitamin B₆ may increase blood levels of homocysteine. Studies have shown that the reverse is also true. It was recently reported that vitamin B₁₂ and folic acid supplements decreased homocysteine levels in subjects with vascular disease and in young adult women, with the most significant drop in homocysteine levels being seen when folic acid was taken alone (Bronstrup, A. et al. “Effects of folic acid and combinations of folic acid and vitamin B₁₂ on plasma homocysteine concentrations in healthy, young women.” Am J Clin Nutr 1998; 68: 1104-10; Clarke, R. “Lowering blood homocysteine with folic acid based supplements. Brit Med J 1998; 316: 894-98). It has also been reported that a significant decrease in homocysteine levels occurred in older men and women who took a multivitamin/multimineral supplement for 8 weeks (McKay, D. et al. “Multivitamin/mineral Supplementation Improves Plasma B-Vitamin Status and Homocysteine Concentration in Healthy Older Adults Consuming a Folate-Fortified Diet.” J.Nutrition 200; 130: 309-96).

A question has been raised as to whether homocysteine levels correlate with actual disease, disease risk or are simply a marker reflecting an underlying process such as oxidative stress which is responsible for both high homocysteine levels and the development of disease. (Seshadri, S. “Elevated Plasma Homocysteine Levels: Risk Factor or Risk Marker for the Development of Dementia and Alzheimer's Disease”. J. Alzheimer's Dis. August 2006; Vol. 9, No. 4: 393-398.) Furthermore, McCaddon et al. note that these mechanisms are not necessarily mutually exclusive—for example, elevated homocysteine levels may perhaps be both a cause and consequence of oxidative stress (McCaddon et al. “Functional Vitamin B₁₂ deficiency and Alzheimer's Disease. Neurology 2002; 58 (9): 1395-99).

It is well-accepted that many vitamin B₁₂-related conditions, regardless of cause, can be easily (and reversibly) treated by administering vitamin B₁₂ or its hydroxycobalamin derivative, either orally or by injection into muscle tissue. As suggested by the above studies, vitamin B₁₂ may also play a role in conditions associated with oxidative stress by decreasing levels of homocysteine or other reactive species.

Thiolatocobalamins may also present useful therapeutic alternatives to vitamin B₁₂ or hydroxycobalamin administration or supplementation. McCaddon and coworkers suggested that GSCbl and related thiolatocobalamins might be more effective than currently available pharmaceutical B₁₂ forms (CNCbl and hydroxycobalamin) in treating of conditions associated with oxidative stress such as Alzheimer's disease (AD) and other neurological diseases (McCaddon, A., Regland, B., Hudson, P.; Davies, G. Neurol 2002; 58: 1395-1399). Numerous studies show that oxidative stress is an important neurodegenerative element in AD and several other neurological diseases. Glutathionylcobalamin is a naturally occurring intracellular form of cobalamin and is more readily absorbed and retained longer than cyanocobalamin. It has been proposed that, in vivo, GSCbl is an intermediate in the conversion of biologically inactive cyanocobalamin to the active coenzyme forms adenosylcobalamin and methylcobalamin. The reducing agent glutathione (GSH) is required for the formation of GSCbl, and is likely to be present in lower levels in AD patients as compared with healthy individuals due to oxidative stress. Thus, GSCbl has the potential to offer a valuable, direct source of cobalamin in therapeutic applications requiring administration of a vitamin B₁₂ derivative. Furthermore, reduced glutathione levels are associated with a wide range of pathophysiological conditions, including liver failure, malignancies, HIV infection, pulmonary disease, and Parkinson's disease. The following list is for example purposes only and, although extensive, is not exhaustive: Acetaminophen poisoning, Attention Deficit Disorder, Autistic Spectrum Disorders, Addison's disease, aging, Acquired Immunodeficiency Syndrome, Amyotrophic lateral sclerosis, ankylosing spondylitis, arteriosclerosis, arthritis (rheumatoid), asthma, autoimmune disease, Behcet's disease, burns, cachexia, cancer, candida, cardiomyopathy, chronic fatigue syndrome, chronic obstructive pulmonary disease, chronic renal failure, colitis, coronary artery disease, cystic fibrosis, diabetes mellitus, Crohn's disease, Down's syndrome, eczema, emphysema, Epstein Barr viral syndrome, fibromyalgia, glaucoma, Goodpasture syndrome, Grave's disease, hypercholesterolaemia, herpes, viral/bacterial/fungal infections, inflammatory bowel disease, systemic lupus erythematosis, senile and diabetic macular degeneration, malnutrition, Meniere's disease, Multiple Sclerosis, Myasthenia Gravis, neurodegenerative diseases, nutritional disorders, pre-eclampsia, progeria, psoriasis, rheumatic fever, sarcoidosis, scieroderma, shingles, stroke, vasculitis and vitiligo.

McCaddon and Davies recently reported on observations concerning the co-administration of N-acetyl-L-cysteine (NAC, a glutathione precursor and potent antioxidant) with B vitamin supplements in cognitively impaired patients, all of whom had high serum homocysteine levels and two of whom had low reported glutathione levels. Improvements in agitation, alertness, and cognitive function were observed in these patients. (McCaddon, A. and Davies, G. “Co-administration of N-acetylcysteine, vitamin B₁₂, and folate in cognitively impaired hyperhomocysteinaemic patients.” Int. J. Geriatr Psychiatry 2005; 20: 998-1000).

McCaddon also reported more recent observations concerning additional hyperhomocysteinanemic patients with cognitive impairment. The case reports demonstrate an apparent clinical efficacy of the addition of 600 mg N-acetyl-L-cysteine (NAC) to B₁₂ and/or folate regimens. (McCaddon, A. “Homocysteine and cognitive impairment; a case series in a General Practice setting.” Nutrtion Journal 2006; 5:6).

In view of the potential benefits reported with the use of naturally occurring glutathionylcobalamin the use of a synthetic version of the compound glutathionylcobalamin and salts of glutathionylcobalamin are of interest as a potential treatment or supplement.

An understanding of the stability of thiolatocobalamins is essential if these compounds are to be used for treatment or supplemental applications. It is also important when exploring the biological relevance of these compounds. A range of thiolatocobalamins have been synthesized, some novel, and studies have been initiated on the stability and reactivity of these compounds as well. Interestingly, the stability of a specific thiolatocobalamin is very dependent on the thiol itself, and can vary over several orders of magnitude.

In efforts to reduce the damaging effects of oxidative stress and to establish the role of thiolatocobalamin treatment or supplementation in conditions associated with oxidative stress, there is a need to identify useful, stable and reactive thiolatocobalamin species. There is also a need not only for simple convenient methods of preparing thiolatocobalamins for use in human and animal studies, but also to develop test protocols that better define the role of oxidative stress (including the effects of reactive species such as homocysteine and hydrogen peroxide) in cell damage. Finally, there is a need to demonstrate the effects of naturally occurring and novel thiolatocobalamins on both healthy cells and those subjected to oxidative stress, including among other things increased homocysteine or H₂O₂ levels, in order to identify useful therapeutic applications for thiolatocobalamins.

### SUMMARY OF THE INVENTION

It has been discovered that glutathionylcobalamin protects cells from damage and death when exposed to oxidative stress conditions. Indeed, glutathionylcobalamin may be useful for a wide range of other diseases associated with oxidative stress. Glutathionylcobalamin may also be useful for dietary supplementation.

The focus of a number of studies has been on the effects of vitamin B₁₂ and its derivatives on homocysteine levels. It is well-recognized that the hallmark of cardiovascular disease, cerebrovascular disease and peripheral vascular disease, among others, is endothelial cell damage. By using cell model data, we have discovered that that glutathionylcobalamin, regardless of whether they are administered alone or in combination with folate, effectively protects endothelial cells against homocysteine-induced oxidative damage. Furthermore, glutathionylcobalamin protects cells against hydrogen peroxide-induced oxidative damage. Importantly, this novel vitamin B₁₂ derivative shows superior protection compared with the currently available pharmaceutical forms of vitamin B₁₂ and folate and naturally occurring cobalamins.

Thus, in accordance with the invention a pharmaceutical composition or dietary supplement respectively comprising glutathionylcobalamin is provided for the treatment of conditions of oxidative stress in animals, including mammals and birds, and specifically including humans; livestock, such as beef and diary cattle, horses, pigs, goats, rabbits and poultry; and domestic animals, such as cats and dogs. The pharmaceutical composition or dietary supplement preferably also comprises a folate composition and a vitamin B₆ composition. The pharmaceutical composition may include additional ingredients as is appropriate for the form of administration, including a pharmaceutically acceptable carrier or solvent.

Further the glutathionylcobalamin of the pharmaceutical composition or dietary supplement may advantageously comprise a crystalline salt of glutathionylcobalamin, and in particular may comprise a biologically accepticalable salt, such as a sodium or potassium salt of said glutathionylcobalamin.

The invention further relates to a method to treat a disease or condition associated with oxidative stress comprising administering an effective amount of glutathionylcobalamin as a biologicallyagent, and preferably of a synthetic glutathionylcobalamin (including a derivative or salt thereof). This agent may be administered in combination with effective amounts of compounds known to reduce serum homocysteine levels, such as one or more of a folate compound and vitamin B6. The disease or condition associated with oxidative stress may be one or more of cardiovascular disease, cerebrovascular disease, peripheral vascular disease, glaucoma, Alzheimer's disease, dementia, and combinations thereof. The invention also relates to a method for the inhibition or reduction of free radical formation comprising administering glutathionylcobalamin (or a derivative or salt thereof, and in particular where the free radical formation is due to high hydrogen peroxide levels, or notably where the free radical is hydrogen peroxide.

### BRIEF DESCRIPTION OF THE DRAWINGS

FIG. 1 is a graphic representation showing the effect of homocysteine and H₂O₂ on endothelial cell viability;

FIG. 2 is a graphic representation showing that NACCbl and GSCbl protect endothelial cells from the effect of homocysteine;

FIG. 3 is a graphic representation showing that NACCbl and GSCbl protect endothelial cells from the effect of H₂O₂;

FIG. 4 is a further graphic representation showing that NACCbl and GSCbl protect endothelial cells from the effect of homocysteine;

FIG. 5 is a further graphic representation showing that NACCbl and GSCbl protect endothelial cells from the effect of H₂O₂;

FIG. 6 is a graphic representation showing that NACCbl and GSCbl protect endothelial cells from apoptosis induced by homocysteine;

FIG. 7 is a series of plots showing the effect of oxidative stress on Hsp32 and Hsp70 gene expression in SK-HEP-1 Cells;

FIG. 8 illustrates a gene expression study for oxidative stress of SK-HEP-1 Cells;

FIG. 9 illustrates Hsp32 gene expression as induced by homocysteine and NACCbl;

FIG. 10 illustrates Hsp32 gene expression as inhibited by quercitin;

FIG. 11 illustrates that NACCbl protects endothelial cells from the effect of homocysteine via a mechanism involving Hsp70 and Hsp32;

FIG. 12 also illustrates that NACCbl protects endothelial cells from the effect of homocysteine;

FIG. 13 illustrates the effect of high concentrations of NACCbl and GSCbl on SK-HEP-1 cells;

FIG. 14 illustrates the effect of the free thiols NA and GSH versus NACCbl or GSCbl in the absence or presence of folate on protecting SK-HEP-1 cells from Hcy;

FIG. 14a illustrates the effects of cobalamins in the presence of folate on protecting T SK-HEP-1 cells from Hcy;

FIG. 15 illustrates the effect of variable Hcy, protection with NACCbl versus Hcy and N-acetyl-cysteine versus Hcy;

FIG. 16 illustrates the normalized raw statistical data normalized to Log 2;

FIG. 17 illustrates the normalized raw statistical data normalized to Log 10;

FIG. 18 illustrates percentage survival rate of U937 cells at a variable concentration of Hcy with the administration of various compositions; and

FIG. 19 illustrates percentage survival rate of Jurkat cells at a variable concentration of Hcy with the administration of various compositions.

### DESCRIPTION OF THE INVENTION

This invention relates to a novel synthetic thiolatocobalamin, glutathionylcobalamin which can be used to protect cells against oxidative stress damage. This composition relate to a pharmaceutical composition or a dietary supplement which advantageously further comprises a folate or folate compound which is used here to includes folate and any natural isomer of reduced folate, such as (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and their polyglutamyl derivatives as described in U.S. Pat. No. 5,997,915. A “pharmaceutical composition” is used herein to mean a composition in a biologically acceptable carrier as is appropriate for the means of administration and at a concentration to provide an acceptable dosage for the intended therapeutic or prophalatic result. A “dietary supplement” is used herein to mean a form that can be acceptably administered as a supplement to the customary dietary intake of the subject animal such as, for example, multivitamin preparations (with or without minerals and other nutrients); breakfast foods such as prepared cereals, toaster pastries and breakfast bars; infant formulas; dietary supplements and complete diet and weight-loss formulas and bars; animal feed (for example pet foods) and animal feed supplements (such as for poultry feed). The amount of the natural isomer of a reduced folate in a composition for human consumption can range between about 5% and about 200% of the daily requirement for folic acid per serving or dose. The animals to which the compositions can be applied for therapeutic effect are advantageously birds or mammals, such as livestock, domestic animal or most advantageously humans.

The invention further relates to a method of treatment of diseases of conditions related to oxidative stress comprising administering a therapeutically effective amount of a composition comprising glutathionylcobalamin (meaning specifically glutathionylcobalamin, its derivatives and salts thereof, preferably with one or more or of a folate compound (as previously discussed) and vitamin B₆. The term “therapeutically effective amount” as used herein refers to an amount of an “glutathionylcobalamin” sufficient to affect the symptoms due to oxidative stress, or free radical presence or inhibit free radical formation to a statistically significant degree. The term “effective amount” therefore includes, for example, an amount sufficient to prevent or treat a condition of oxidative stress, such as dementia or stroke. The dosage ranges for the administration of glutathionylcobalamin are those that produce the desired effect. Generally, the dosage will vary with the age, weight, condition, and sex of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the extent of oxidative conditions or diseases by methods well known to those in the field. Moreover, the glutathionylcobalamin can be applied in pharmaceutically acceptable carriers known in the art. The glutathionylcobalamin can be used to treat conditions or diseases associated with oxidative stress in animals and in humans in vivo. The application can be oral, by injection, or topical, providing that in an oral administration the glutathionylcobalamin is preferably protected from digestion.

The glutathionylcobalamin may be administered to a patient by any suitable means, including oral, parenteral, subcutaneous, intrapulmonary, topically, and intranasal administration. Parenteral infusions include intramuscular, intravenous, intraarterial, intraperitoneal or intravitreal administration. The glutathionylcobalamin may also be administered transdermally, for example in the form of a slow-release subcutaneous implant, or orally in the form of capsules, powders, or granules. Although direct oral administration may cause some loss of activity, the glutathionylcobalamin could be packaged in such a way to protect the active ingredient(s) from digestion by use of enteric coatings, capsules or other methods known in the art.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The glutathionylcobalamin may be formulated into therapeutic compositions as pharmaceutically acceptable salts. These salts include the acid addition salts formed with inorganic acids such as, for example, hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of the glutathionylcobalamin may be controlled by altering the concentration of the macromolecule.

Another method for controlling the duration of action comprises incorporating the glutathionylcobalamin or a salt or derivative thereof into particles of a polymeric substance such as a polyester, peptide, hydrogel, polylactide/glycolide copolymer, or ethylenevinylacetate copolymers. Alternatively, the glutathionylcobalamin may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The present invention provides a method of preventing, treating, or ameliorating a disease that results from development of oxidative stress in the body, such as cardiovascular disease, neural tube defects, osteoporosis, stroke and other cerebrovascular disease, peripheral vascular disease, glaucoma, Alzheimer's disease and dementia, comprising administering to a subject at risk for a disease or displaying symptoms for such disease, an effective amount of the glutathionylcobalamin I. The present invention also provides a method of preventing, treating, or ameliorating a disease that results from an increase in free radical activity, such as inflammation, oxidative stress, rheumatoid arthritis, aging, arthrosclerosis, multiple sclerosis, asthma, inflammatory bowel disease, chronic inflammatory demyelinating polyradioculoneuritis, and cancer. The term “ameliorate” refers to a decrease or lessening of the symptoms or signs of the disorder being treated. The symptoms or signs that may be ameliorated, for example, include those associated with dementia or AD.

For purposes of the inventions described herein, the structure and purity of the synthetic compound glutathionylcobalamin was characterized using UV/Vis Spectrophotometry, ¹H NMR spectroscopy, X-ray crystallography, XAS (spectrum not shown) and ES-MS (data not shown) for purposes of providing a thorough characterization of the new compound and evaluating its purity, stability and reactivity. The synthesis and characterization of glutathionylcobalamin has been previously reported in U.S. Pat. No. 7,030,105, the entire contents of which are incorporated herein by reference. %). The synthesis is carried out in aqueous solution by the addition of a small excess of thiol to a highly concentrated solution of aquacobalamin, followed by the addition of acetone to precipitate the product after completion of the reaction. The aqueous solvent is water alone. Where the reaction is carried out in a mixture of water and water miscible solvent, the proportion of water to water miscible solvent may depend on the kinetics and/or thermodynamics of the reaction. The reaction mixture may also optionally contain additional agents such as buffers, for example, MES. The resultant cobalamin derivatives may be slightly light-sensitive, therefore, preferably, the reaction is carried out under red light only conditions.

The reaction may be performed at a temperature from 0° to about 60°. The reaction may The reaction is performed in an aqueous solvent, being water alone or a mixture of water and a water miscible solvent (such as MeOH, EtOH, PrOH & BuOH). Preferably be carried out at ambient room temperature, such as from about 15° C. to about 30° C., for example about 20-25° C. The reaction is allowed to proceed for a time sufficient to achieve substantial completion. Reference to substantial completion of the reaction is intended to refer to the substantial consumption (e.g. greater than 95%) of the HOCbl•HX. Precipitation of the resultant products may be performed under cooling, for example ice cooling, e.g. to about −10-10° C. However, yield of the cobalamin products can be increased by the addition of a precipitate inducing solvent. The precipitate inducing solvent used to precipitate the formed product which is preferably a water miscible solvent less polar then water and includes alcohols (such as MeOH, EtOH, PrOH & BuOH) and acetone, is added in an amount sufficient to induce precipitation of the formed glutothionyllcalamin. A preferred precipitate inducing solvent is acetone. In a preferred embodiment of the invention, the reaction is carried out with from 1.1 to about 2.5 equivalents of hydroxycobalamin, (“GluSH”), more preferably from about 1.2 to about 2 equivalents. Further in an alternative embodiment, the amount of hydroxycobalamin approaches saturation in the aqueous solvent, or in yet again another alternative embodiment, the reaction is run with a slight excess of GluSH and a concentration of at least about 0.025M hydroxycobalamin in the aqueous solvent is used. The synthesis is carried out under red light only conditions. Hydroxycobalamin hydrochloride (HOCbl•HCl, 98% (stated purity by manufacturer) was purchased from Fluka. The percentage of water in HOCbl•HCl (•nH₂O) (12±2%) was determined by converting HOCbl•HCl to (CN)₂Cbl- and the concentration of (CN)₂Cbl- determined by UV-vis spectroscopy (Barker, H. A.; Smyth, R. D.; Weissbach, H; Toohey, J. I.; Ladd, J. N.; Volcani, B. E. J. Biol. Chem., 1960, 235, 480). Glutathione (GluSH, 98%; i.e., in its reduced form) was purchased from Aldrich. ¹H NMR spectra were recorded on an Inova 500 MHz spectrometer equipped with a 5 mm thermostatted (25.0±0.2° C.) probe. All solutions were prepared in D₂O and TSP (3-(trimethylsilyl)propionic-2,2,3,3-d₄ acid, sodium salt) was used as an internal standard. Visible spectra were recorded on a Cary 1E spectrophotometer equipped with a thermostatted cell changer (25.0±0.1° C.) and operated with WinUV Bio software (version 2.00).

According to this method of synthesis, a final product with greater than 90% purity, preferably greater than about 95% purity, more preferably 97, 98 or 99% purity as determined by the any of methods described herein such as, for example, ¹H NMR spectroscopy or the dicyanocobalamin test described by Barker et al., J. Biol. Chem. 1960, 135, 181-190 incorporated herein by reference as if fully set forth herein. The precipitated Na[NACCbl] is collected by filtration, preferably under suction, and optionally washing the precipitate with a suitable solvent or mixture of solvents such as acetone and/or ether. In another embodiment of the invention, the precipitate can be collected by decanting off the solvents or removing them by suction. Preferably, the precipitate is further dried to remove any remaining solvent. This may be carried out by under vacuum, optionally with heating (at a temperature which does not decompose the Na[NACCbl], for example from about 25-40° C.). The hydrochloride salt of hydroxycobalamin, HOCbl•HCl (81.13 mg, 5.16×10⁻⁵ moles (12% H₂O)) was dissolved in distilled water (0.800 ml) in a vial with gentle heating i.e. using a heat gun on a low setting. At these concentrations the solution is an intense red color and quite thick in appearance. After the addition of 0.382 ml of GluSH (0.261 M, 9.97×10⁻⁵ moles), the vial was capped, vigorously shaken and left in the dark for 3 hr. A purple precipitate formed upon the addition of 1.00 ml acetone. After cooling in an ice bath for 30 min, the purple precipitate was filtered under suction (water aspirator), washed with acetone and ether and dried at 50° C. for 2 days under vacuum (0.13 mbar). Yield 63.6 mg (78%). Duplicate syntheses gave yields of 76 and 86%.

Structure

UV/Vis spectrophotometry can be used to characterize thiolatocobalamins. All spectra were recorded using a Varian Cary 5000 spectrophotometer. Data in Table 1 below showed that all thiolatocobalamins have a similar electronic spectrum with characteristic bands at 333, 372, 428 and 534 nm that are in agreement with previous reports for other thiolatocobalamins.

TABLE 1

| | |
|---|---|
| Cbl | γ max (nm) |

| | | | | |
|---|---|---|---|---|
| GSCbl | 333 | 372 | 428 | 534 |
| NACCbl | 333 | 372 | 428 | 534 |
| HcyCbl | 333 | 372 | 428 | 534 |
| NACMECbl | 333 | 372 | 428 | 534 |

The ¹H NMR spectrum of the cobalamins was also recorded (500 MHz Varian spectrometer, D₂O, 25° C.). NACMECbl IS 2-N-acetylamino-2-carbomethoxy-L-ethanethiolatocobalamin. Thiolatocobalamins have five characteristic signals in the aromatic region (B₇, B₂, B₄, R₁ and C₁₀ protons, see FIG. 1 for assignment). Table 2 below summarizes the results. It can be seen that all thiolatocobalamins have similar chemical shifts, and they are in agreement with reported values.

TABLE 2
¹H NMR Data

| | Chemical Shift (ppm) | | | | |
|---|---|---|---|---|---|
| Cobalamin | B₇ | B₂ | B₄ | R₁ | C₁₀ |
| GSCbl | 7.19 | 6.95 | 6.39 | 6.28 | 6.09 |
| NACCbl | 7.19 | 6.95 | 6.40 | 6.28 | 6.09 |
| HcyCbl | 7.20 | 6.95 | 6.38 | 6.28 | 6.10 |
| NACMECbl | 7.19 | 6.95 | 6.40 | 6.28 | 6.09 |

Purity

The purity of the products was also assessed by ¹H NMR spectroscopy and by the dicyanocobalamin test. All B₁₂ derivatives are converted to dicyanocobalamin ((CN)₂Cbl) upon the addition of cyanide; hence, the percentage of non B₁₂ impurities can be determined. Table 3 below shows the results obtained.

TABLE 3
Purity Assay

| | | ¹H NMR |
|---|---|---|
| Cobalamin | (CN)₂Cbl Test | Spectroscopy |
| GSCbl | 99% | 98% |
| NACCbl | 95% | 98% |
| HcyCbl | 97% | 98% |
| NACMECbl | 96% | 98% |

X-Ray Diffraction Studies

Crystals of Na[NACCbl] 18H₂O were grown in water. Diffraction experiments were carried out on beamline BL11-1 at the Stanford Synchrotron Radiation Laboratory (SSRL). Data were collected on an ADSC Q-315 CCD detector using X-rays produced by a 26 pole wiggler insertion device, with a wavelength of 0.81798 Å (15160 eV) from a side scattering bent asymmetric cut Si (111) crystal monochromator. Table 4, below, shows bond length data for NACCbl. Bond length data for γ glutamylcysteinylcobalamin (γ-GluCysCbl) is also given for comparison purposes.

TABLE 4

| | NACCbl | γ-GluCysCbl | Δ(NACCbl-γGluCysCbl) |
|---|---|---|---|
| Co—S | 2.25 | 2.27 | −0.02 |
| Co—NB₃ | 2.06 | 2.05 | +0.01 |
| Co—N₂₁ | 1.88 | 1.89 | −0.01 |
| Co—N₂₂ | 1.92 | 1.90 | +0.02 |
| Co—N₂₃ | 1.93 | 1.91 | +0.02 |
| Co—N₂₄ | 1.88 | 1.89 | −0.01 |

Stability Studies

The decomposition of GSCbl, NACCbl, and HcyCbl in PBS at 37° C. was monitored by UV/Vis spectrophotometry. Table 5 below shows t_{1/2} and observed rate constant, k_{obs}, calculated for each derivative.

TABLE 5

| | t_{1/2} (hr) | K_{obs} (min⁻¹) |
|---|---|---|
| GSCbl | No decomposition within 20 hours | |
| NACCbl | No decomposition within 20 hours | |
| HcyCbl | 6.6 | 0.0018 |

After characterization, NACCbl was subjected to several experiments to determine if it offered protection to endothelial and other cells subject to oxidative stress under variable concentrations of homocysteine or H₂O₂. Experiments were also conducted to determine what, if any, detrimental effects NACCbl and GSCbl have on endothelial cells at increasing concentrations. While not wishing to be bound by any specific theory, experiments were conducted to determine potential mechanisms by which NACCbl's protective effects occur. Finally, experiments were conducted to determine if NACCbl offered any advantage in protection over other thiolatocobalamins, cobalamins or folate. These experiments are set forth in the examples below.

### EXAMPLES

### Reagents

The conduct of the experiments required a number of reagents, which are set forth below. However, the experiments are not limited to the specific reagents listed, and other reagents, useful in the described methods, are well within the spirit and intention of the invention.

### Reagents/Materials Used in Experiments

The reagents, assays, kits and other materials used in the experiments are set forth in the lists below. All chemicals were obtained from Sigma-Aldrich Company Limited, Poole, Dorset, UK, unless otherwise indicated.

### Reagents

MegaCell® M.E.M. Media. Sigma. Product Code: M4067
DMEM without L-glutamine and phenol red. BioWhittaker, Cambrex Bioscience, Nottingham, UK.
FetalClone 1 Serum; Triple 0.1 μM filtered. HyClone, Logan, Utah, USA. Cat No. SH30080.03
5-Methyltetrahydrofolic acid disodium salt: [5-Methyl-5,6,7,8-tetrahydropteroyl-L-glutamic acid disodium salt]; C₂₀H₂₃N₇Na₂O₆; F.W. 503.42; EC No. 2.1.1.13
DL-Homocysteine: [2-Amino-4-mercaptobutyric acid] HSCH₂CH₂CH(NH₂)COOH; F.W. 135.18; EC No. 207-222-9
Dimethyl sulfoxide: [DMSO, Methyl sulfoxide]; (CH₃)₂SO; F.W. 78.13; EC No. 200-664-3
Pyridoxine: [Pyridoxol; Vitamin B₆]; C₈H₁₁NO₃; F.W. 169.18; EC No. 200-603-0
Vitamin B₁₂: [CN-Cbl; Cyanocobalamin]; C₆₃H₈₈CoN₁₄O₁₄P; F.W. 1355.37; EC No. 200-680-0
Methylcobalamin: C₆₃H₉₁CoN₁₃O₁₄P; F.W. 1344.38; EC No. 236-535-3
Vitamin B₁₂ a: [Aquocobalamin chloride]; C₆₂H₉₀CICₒN₁₃O₁₅P; F.W. 1382.82; EC No. 261-200-3
Hydroxocobalamin: [Hydroxocobalamin acetate salt, Vitamin B₁₂a]; C₆₄H₉₁CoN₁₃O₁₆P; F.W. 1388.39; EC No. 236-533-2
Bilirubin Mixed Isomers [Bilirubin IX-alpha] C₃₃H₃₆N₄O₆; M.W. 584.68; EC No. 211-239-7

Glutathionylcobalamin: GSCbl M.W. 1635.0 synthesized by the inventors as described in U.S. Pat. No. 7,030,105

N-acetyl-L-cysteinylcobalamin: NACCbl M.W. 1491.0 synthesized by the inventors
Quercetin dehydrate: [2-(3,4-Dihydroxyphenyl)-3,5,7-trihydroxy-4H-1-benzopyran-4-one dihydrate]; C₁₅H₁₀O₇.2H₂O; F.W. 338.27; EC No. 204-187-1
Sn Protoporphyrin(IX) dihydrochloride
   8,13-Bis(vinyl)-3,7,12,17-tetramethyl-21H, 23H-porphine-2,18dipropionic acid tin(IV) dichloride; [Sn(IX) PP]; C₃₄H₃₂N₄O₄SnCl₂; M.W. 750.26; Frontier Scientific Inc., Carnforth, UK.
Hydrogen peroxide solution: 30% (w/w): H₂O₂: F.W. 34.01; EC No. 231-765-0
Propidium iodide: [3,8-Diamino-5[3-(diethylmethylammonio)propyl]-6-phenyl-phenanthridinium diiodide]; C₂₇H₃₄I₂N₄; M.W. 668.41; EC No. 247-081-0
Necrosis Inhibitor: IM 54; [2-(1H-Indol-3-yl)-3-pentylamino-maleimide]; C₁₉H₂₃N₃O₂; M.W. 325.4; Cat No. 480060
Etoposide: [VP-16]; C₂₉H₃₂O₁₃; M.W. 588.6; EC No. 251-509-1; Calbiochem, Nottingham, UK.
Hemin: C₃₄H₃₂C₁FeN₄O₄; M.W. 651.96; EC No. 240-140-1
Trypan Blue: [Direct Blue 14]; C₃₄H₂₄N₆Na₄O₁₄S₄; F.W. 960.81; EC No. 200-786
Z-VAD-FMK: C₂₂H₃₀O₇N₃F; M.W. 467.5
Trypsin-EDTA solution 0.25%: 0.25%, 2.5 g porcine trypsin, 0.2 g EDTA; M.W. 23.8 kDa; EC No. 3.4.21.4
N-Acetyl-L-Cysteine: [LNAC; NAC]; HSCH₂CH(NHCOCH₃)CO₂H; F.W. 163.19; EC No. 210-498-3
_{L}-Glutathione reduced: (γ-Glu-Cys-Gyl; CSH); [γ-_{L}-Glutamyl-_{L}-cysteinyl-glycine]; H₂NCH(CO₂H)CH₂CH₂CONHCH(CH₂SH)CONHCH₂CO₂H; F.W. 307.32; EC No. 200-725-4
   Assays and Kits
EnzoLyte™ Rh110 Caspase-3 Assay Kit; AnaSpec, San Jose, Calif., USA; Cat No.
CellTiter® Aqueous One Solution Cell Proliferation Assay; Promega Corporation, Madison, Wis., USA.
   RT-PCR Reagents
QuickPrep micro mRNA Purification Kit; Amersham Pharmacia Biotech; Cat. No. 27-9255-01; Buckinghamshire, UK.
Ready-To-Go You-Prime First-Strand Beads; Amersham Pharmacia Biotech; Cat. No. 27-9261-01; Buckinghamshire, UK.
puReTaq™ Ready-To-Go™ PCR Beads; Amersham Biosciences; Cat. No. 27-9558-01; Piscataway, N.J. USA.
BenchTop 100 bp DNA Ladder; Promega Corporation, Madison, Wis., USA; Cat. No. G8291.
Trackit™ 100 bp DNA Ladder: 0.1 μg/μl; Invitrogen; Cat. No. 10488-058; Paisley, UK.
Agarose 1 Biotechnology Grade; Amresco, Solon, Oh., USA; Product No. 0710-500 G
Ethidium Bromide Fluorescence λ ex 530 nm; λ em 600 nm; [3,8-Diamino-5-ethyl-6-phenylphenanthridinium bromide]; C₂₁H₂₀BrN₃; F.W. 394.32; EC No. 1239-45-8; Appligene Oncor, Graffenstaden, Germany.
DAPI: [4′,6-Diamidino-2-phenylndole,dilactate]; C₂₂H₂₇N₅O₆; F.W. 457.48
Pd(N)₆ Sodium salt; Amersham Pharmacia, N.J. USA; Cat No. 27-2166-01
Primers for RT-PCR: All primer sequences obtained from Alta Biosciences University of Birmingham, UK.
   siRNA Reagents
Lipofectamine™ 2000
   3:1 (w/w) liposome formulation of the polycationic lipid 2,3-dioleyloxy-N-[2(sperminecarboxamido)ethyl]-M, N-dimethyl-1-propanaminium trifluoroacetate (DOSPA), and the neutral lipid dioleoyl phosphatidylethanolamine (DOPE) in membrane filtered water. Invitrogen, Paisley, UK; Cat No. 11668-019
DNase-, RNase- protease-free Water DEPC treated. Dihydrogen oxide H₂O Mᵣ 18.02 filtered 0.2 μM membrane. EC No. 231-791-2; BioChemika, Fluka, Poole, UK.
RNase Removing Solution Biotechnology Grade; Amresco, Solon, Ohio. USA; Cat No. 6440
OptiMem1 Media; Invitrogen. Paisley, UK; Cat No. 31985-062
siControl RISC-Free siRNA; Dharmacon Inc. Boulder, Colo. USA; Cat No. D-001220-01-20
siControl Non-Targeting siRNA #1; Dharmacon Inc. Boulder, Colo. USA; Cat No. D-001210-01-20
siControl Tox Transfection Control; Dharmacon Inc. Boulder, Colo. USA; Cat No. D-001500-01-20
Individual siRNA Primer Sequences; Dharmacon Inc, Boulder, Colo. USA.
   Cell Lines
A. SK-HEP-1 Cells; ECACC (European Collection of Cell Cultures); No. 91091816
   Cell Line Name
   SK-HEP-1 Human liver adenocarcinoma
   Cell Line Description
      Derived from an ascites sample from a 52 year old male suffering from adenocarcinoma of the liver. The cells have now been shown to be endothelial in origin (In Vitro 1992; 28A: 136).
   Species:Human. Tissue:Liver. Morphology:Endothelial
   Sub Culture Routine
      Split sub-confluent cultures (70-80%) 1:2 to 1:4 i.e. seeding at 2-4×10,000 cells/cm² using 0.25% trypsin or trypsin/EDTA; 5% CO₂; 37° C.
   Karyotype: Hyperdiploid to hypotriploid
B. U937 (monocytes)—Purchased from ECACC and cultured under known standard conditions.
C. Jurkat (T-cells)—T-lymphocyte leukemic cell line, purchased from ECACC and cultured under known, standard conditions.
   Gene Accession
   Heme Oxygenase-1 (decyclizing) Human EC 1.14.99.3
      Reaction: heme+3AH₂+3O₂=biliverdin+Fe²+CO+3A+3H₂O
      Swiss-Prot: P09601 Gene Name: HMOX1 Location: Microsomal Sequence Information Length 288 M. M.W. 32819 Da. Chromosome 22q12
   Heat Shock 70 kDa Human
      Swiss-Prot: P17066 Gene Name: HSPA6 Location: Cytosolic
      Sequence Information Length 643AA. M.W. 71028 Da. Chromosome 1q23
   Transcription regulator protein BACH1 Human
      Swiss-Prot: 014867 Gene Name: BACH-1 Location: Nucleus
      Sequence Information Length 736 M. M.W. 81958 Da. Chromosome 21q22.11
   78 kDA glucose-regulated protein Human
      Swiss-Prot: P11021 Gene Name: HSPA5: GRP78 Location: Endoplasmic reticulum.
      Sequence Information Length 654 M. M.W. 72333 Da. Chromosome 9q33-q34.1
   Heat-shock protein beta-1 Human
      Swiss-Prot: P04792 Gene Name: HSP27 Location: Cytoplasm, translocates to nucleus during heat-shock.
      Sequence Information Length 205AA M.W. 22783 Da. Chromosome 7q 11.23
   Heat shock protein HSP 90-alpha Human
      Swiss-Prot: P07900 Gene Name: HSP90A Location: Cytoplasm
      Sequence Information Length 731M. M.W. 84529 Da. Chromosome 14q32.33
   Heat shock protein HSP 90-beta Human
      Swiss-Prot: P08238 Gene Name: HSP90B Location: Cytoplasm
      Sequence Information Length 723M. M.W. 83133 Da. Chromosome 6q12
   Beta-actin, cytoplasmic 1 Human
      Swiss-Prot: P60709 Gene Name: ACTB Location: Cytoplasm
      Sequence Information Length 375M. M.W. 41737 Da. Chromosome 7q22.1
   94 kDa Glucose-regulated protein Human
      Swiss-Prot: P14625 Gene Name: GRP94 Location: Endoplasmic reticulum

Sequence Information Length 803AA. M.W. 92469 Da. Chromosome 12q23.3

RT-PCR Primer Sequences.

| | |
|---|---|
| Sense Primer | Antisense Primer |

| | | |
|---|---|---|
| β- | TGC-TAT-CCC-TGT- | AGT-ACT-TGC-GCT-CAG-GAG-GA |
| actin | ACG-CCT-CT | |
| Hsp 27 | ATG-GCG-TGG-TGG- | CAA-AAG-AAC-ACA-CAG-GTC-GC |
| | AGA-TCA-CC | |
| HO-1 | CAG-GCA-GAG-AAT- | GCT-TCA-CAT-AGC-GCT-GCA |
| | GCT-GAG-TTC | |
| Hsp 70 | TTC-CGT-TTC-CAG- | CGT-TCA-GCC-CCG-CGA-TGA-CA |
| | CCC-CCA-ATC | |
| Hsp | AGA-AGG-TTG-AGA- | AAG-AGT-GAG-GGA-ATG-GG |
| 90β | AGG-TGA-CAA | |
| grp 78 | GAT-AAT-CAA-CCA- | GTA-TCC-TCT-TCA-CCA-GTT-GG |
| | ACT-GTT-AC | |
| gp 96 | TGC-CAA-GGA-AGG- | GTT-GCC-AGA-CCA-TCC-GTA-CT |
| | AGT-GAA-GT | |
| BACH-1 | GGA-CAC-TCC-TTG- | TGA-CCT-GGT-TCT-GGG-CTC-TCA |
| | CCA-AAT-GCA | |

Primer Sequences Used for RT-PCR.
DNA sequences are indicated from 5′ to 3′ terminus according to convention. Hsp and grp 78 primers from Wang et al. (1999).
HO-1, gp 96, β-actin and BACH-1 primers designed from gene sequences obtained from the NCBI website, (http://www.ncbi.nlm.nih.gov/).
Primers were designed using Primer 3 software (http:/www-genome.wi.mit.edu/cgi-bin/primer/primer3www.cqi).
   Experimental Methods

The methods used for cell preparation and the various tests are set forth in detail below. The reagents, including assays, kits, and cell lines are described above.

### Cell Lines

The SK-HEP-1 (ECACC No. 91091816) is a human liver adenocarcinoma cell line. It is derived from an ascites sample from a 52 year old male human suffering from adenocarcinoma of the liver. The cells have now been shown to be endothelial in origin. SK-HEP-1 cells are very sensitive to homocysteine. Jurkat (T-cells) and U937 (monocytes) cell lines were also used. Jurkat and U937 cells are more resistant to homocysteine than SK-HEP-1 cells, but do demonstrate adverse effects when exposed to homocysteine.

### Preparation of Cell Culture Media

Megacell MEM media was supplemented with 3% serum (Fetalclone® 1) and 200 mM L-glutamine. Aliquots (2 ml) were regularly transferred to a 24-well plate and examined under a light microscope for infections and integrity of the culture media.

### Cell Culture

Cells were subcultured 1.2 in culture flasks or seeded into various plates as required for experimental use. For passaging of SK-HEP-1, the medium was removed and the cells were washed with serum free medium. After addition of 1 ml or 2 ml of trypsin/EDTA 0.4% solution per 25 cm² or 75 cm² flask, respectively, the trypsin was removed after 90 seconds. The digestion was stopped after a further 3 minutes by the addition of 5 ml of fresh complete medium. For experimental use, cells between passage three and fifteen were grown in a monolayer until approx. 90% confluent in 6-, 12-, 24-, 48-, or 96-well plates. For siRNA experiments, cells between passage three and ten were grown until approximately 60-70% confluence was reached in 6-, 12-, 24-, or 48-well plates. The cells were allowed to adhere to the plastic surface of the culture vessels for a period of 24 hours prior to experimentation. Cells were grown at 37° C. in a 5% CO₂ humidified Heracell incubator.

### Determination of Cell Counts and Viability

Routine evaluation of the quality and growth rate of cultured SK-HEP-1 cells was accomplished by use of an inverted phase-contrast microscope at 100× magnification. Endothelial cells display “cobblestone” morphology at confluence. After prolonged maintenance at full confluence, these cells may acquire a ‘sprouting’ phenotype and infiltrate under other cells. Characteristics of endothelial cells include a flat irregular shape, multiple small vesicles, and pleiomorphic oval nuclei and are approximately 10-20 μm in diameter.

### Trypan Blue Exclusion Test of Viability

Regular cell counts were performed and cells were stained with Trypan Blue to determine viability and cell counts. Cells suspended in media were diluted 1:1 with Trypan Blue and incubated for 20 minutes. A cell count was performed using 20 μl of this suspension with a haemocytometer according to the manufacturer's instructions.

### Freezing, Storage and Thawing of SK-HEP-1 Cells

For long-term storage, confluent SK-HEP-1 cells were detached with trypsin/EDTA 0.4% solution. Cells were transferred to a centrifuge tube and centrifuged at 218 g for 3 minutes. The culture media was removed and the cell pellet was resuspended in 1.0 ml of freeze media (complete MegaCell media supplemented with 10% [v/v] sterile DMSO). The cell suspension was transferred to cryovials and frozen immediately at −20° C. for 24 hours, then at −80° C. for 7 days and then transferred to −96° C. in vapour phase liquid nitrogen. This procedure was performed in order to ensure gradual freezing of the cells to avoid ice-crystal formation within the cell structure. For thawing of cells, SK-HEP-1 cells were warmed quickly in a 37° C. water bath and the cell suspension was immediately transferred to a 25 cm² cell culture flask containing 9 ml fresh complete MegaCell MEM media. Cells were grown at 37° C. in a 5% CO₂ humidified Heracell incubator.

### Sterilization of Equipment

Filter units containing 0.2 μm filters were autoclaved for filter sterilization of all reagents used under experimental conditions in culture media.

### Cell Treatments

Cells were plated on a 96-well plate at approximately ±5000 cells per well and cultured for 24 hours. Then, subconfluent cells were exposed to test treatments for times indicated. Media was removed from test wells and replaced with 100 μl phenol-red free media containing various concentrations of test compounds. Plates were incubated for either 2 hours or 24 hours.

### MTS Assay

MTS® assay is a standard measure of cell activity. The CellTiter 96® Aqueous One Solution Cell Proliferation Assay was used according to the manufacturer's instructions. Reduction of the MS tetrazolium compound to formazan was detected by color development at 490 nm using a Bio-Tek Synergy H. T. Multi-Detection Microplate Reader, running KC-4 v 3.4 software. After treatment, all media was removed and 100 μl of fresh media was added to each well. 20 μl of the CellTiter 96® solution was added to each test well, and the plate was further incubated for 3 hours.

### Gene Expression Experiments

Cells were plated into 6-, 12-, or 24-well cell culture clusters, at 2×10³, 2×10⁵, or 1×10⁵ cells per well and incubated until 90% confluent. Treatments were applied as above.

Following incubation, cells were subjected to mRNA extraction followed by cDNA synthesis for each sample under test. Polymerase chain reaction cDNA templates were prepared for simplex PCR protocol. PCR products were then visualized using a UV Transilluminator and images captured using a Kodak ID gel imaging system. Densitometry and statistical analysis was then performed on each gene expression band image using PSP® v 10.0 running under Windows XP®.

### Preparation of mRNA from Cell Cultures

mRNA extraction was performed using the Quickprep micro mRNA Purification Kit. Following incubation of cells post-treatment, the media was removed from the cells, adherent cells were re-suspended in 0.4 ml extraction buffer and 0.8 ml elution buffer at 65° C. was added.

Cell suspension was mixed and transferred to a 1 ml microcentrifuge tube. For each sample, 1 ml of oligo(dt)-cellulose was added to a separate microcentrifuge tube. The cell suspension and oligo(dt)-cellulose were centrifuged for 2 minutes at 15,130 g. Supernatant from the oligo(dt)-cellulose was removed and discarded.

Subsequently, 1 ml of cleared homogenate from cell suspensions was added to the pelleted oligo(dt)-cellulose. The sample was re-suspended by inversion for 3 minutes and further mixed in a WhirliMixer for 30 seconds. This mixing step allows binding to occur between the poly-A-tail of the mRNA and the T bases on the oligo-(dt)-cellulose. The oligo(dt)-cellulose was pelleted by centrifugation at 15,130 g for 10 seconds. The supernatant was then discarded.

Each sample was further re-suspended in 1 ml of HIGH salt buffer, the oligo(dt)-cellulose containing cell sample was pelleted by centrifugation at 15,130 g for 10 seconds.

This HIGH salt washing step was carried out a further four times to remove cell debris.

Each sample was then re-suspended in 1 ml LOW salt buffer, and oligo(dt)-cellulose pelleted by centrifugation at 15,130 g for 10 seconds. The supernatant was discarded. This LOW salt washing step was carried out three times in total.

Each sample was then re-suspended in 0.5 ml LOW salt buffer and the slurry transferred to a clean microcentrifuge tube containing a spin column. Samples were centrifuged at 15,130 g for 5 seconds. The eluant was discarded and 0.5 ml LOW salt buffer was carefully added to the spin column. Samples were centrifuged at 15,130 g for 5 seconds. This final step was repeated three times in total.

The spin columns were transferred to clean microcentrifuge tubes. Pre-warmed elution buffer (0.2 ml) at 65° C. was added to the spin column. Samples were further centrifuged at 15,130 g for 5 seconds. This step elutes the mRNA from the oligo(dt)-cellulose into the microcentrifuge tube. The microcentrifuge tubes were then incubated at 65° C. for ten minutes and then placed on ice to preserve the integrity of the mRNA and to prevent base pairing of the mRNA.

### cDNA Synthesis

A reaction mixture was prepared in individual microcentrifuge tubes containing 2 cDNA synthesis beads, 32 μl of the mRNA solution and 1 μl of pd(N)₆. This mixture was incubated at 37° C. in a water bath for 60 minutes. After incubation, 27 μl of RNase-free DEPC treated water was added to each tube to make a total volume 60 μl. cDNA samples were prepared in duplicate. Sample 1 was used immediately in RT-PCR protocol. Sample 2 was prepared for qPCR protocol by the addition of 150 μl of 95% ice-cold ethanol and stored at −20° C.

### Polymerase Chain Reaction

Microcentrifuge tubes from puReTaq™Ready-To-Go™ PCR Beads containing one PCR bead were labeled for each sample required. Added to the PCR bead were 17 μl of RNase-free DEPC treated water, 1 μl sense primer, 1 μl anti-sense primer and 5 μl of the cDNA solution. RT-PCR conditions were as follows: Hsp70 and β-actin, pre-treatment step, 94° C. for 1 minute, followed by denaturing at 92° C. for 1 minute, annealing at 60° C. for 1 minute and extension at 72° C. for 1 minute. Total cycles, 30, post-treatment was then carried out at 72° C. for 10 minutes. For Hsp32, pre-treatment step, 95° C. for 2 minutes, followed by denaturing at 94° C. for 30 seconds, annealing at 58° C. for 1 minute and extension at 72° C. for 1 minute. Total cycles 45, post treatment was then carried out at 72° C. for 10 minutes. Following RT-PCR, samples were stored at −20° C.

### Gel Electrophoresis of RT-PCR Products

Agarose (0.56 g) and 0.56 ml TAE buffer (50×) was added to 27.44 ml ddH₂O. The solution was brought to boiling point for 40 seconds in a microwave. Then, 10 μl ethidium bromide (concentration 1 mg/ml) was added and the solution swirled to mix. Ethidium bromide intercalates with RNA and therefore allows visualization of the bands under UV light. The gel solution was immediately poured into a casting chamber of the electrophoresis kit containing 8-well combs. The gel was allowed to set at room temperature for 30 minutes. The combs were then removed and 100 ml of agarose running buffer was poured into the casting chamber.

Amplification products were separated on a 1.8% agarose gel (m/v) in TAE buffer. The size of the PCR products was determined by comparison to DNA fragments of a well-defined size; therefore, 5 μl of the DNA Ladder was carefully pipetted into the first well of the gel. Successive 10 μl of each PCR sample was then pipetted into subsequent wells on the agarose gel. The gel was connected to the Power Pack and run at 100V for 30 minutes.

Gels were visualised on a UV Transilluminator. Photographs were stored using a Kodak Digital camera system fitted with a UV filter set connected to a PC. Images were then transferred to the Kodak ID gel imaging system. Densitometry was performed on each gene expression band using PSP™ v10 running under Windows XP®.

### Caspase-3 Activity (Apoptosis Assay)

Caspase 3 is activated when cells undergo apoptosis. Caspase-3 assay is a standard for apoptosis assay. Homocysteine is a known inducer of apoptosis. Cells were cultured in 96-well plates for 24 hours and then treated with reagents under test conditions. Cytotoxic agents, H₂O₂ and etoposide were added to negative control wells. The assay was performed according to the manufacturer's protocol, as follows: Cells were re-suspended in 100 μl of clear media and 50 μl of Caspase-3 substrate solution was immediately added to each test well. Plates were incubated at 37° C. for 60 minutes and formation of free 7-amino-4-trifluoro-methylcoumarin (AFC) was acquired by fluorescence measurement at 496/520 nm by Microplate reader.

### Propidium Iodide Assay (Necrosis Assay)

Propidium iodide is a standard assay for necrosis. H₂O₂ is a known inducer of necrosis. Cells were cultured in 96-well plates for 24 hours and then treated with reagents under test conditions. Cytotoxic agents, H₂O₂ and etoposide were added to negative control wells. Cells were re-suspended in 50 μl of clear media and 50 μl of 5 μg/ml propidium iodide solution was added under red-light conditions as the propidium iodide is light sensitive. Plates were incubated at 37° C. for 20 minutes and then absorbance was measured at λex 535 nm/λem 617 nm by a Microplate reader.

### RNA Interference RNAi

siRNAs for human HO-1 were synthesized in 2′-deprotected, duplexed, desalted and purified form by Dharmacon Research Inc., published sequences from Zhang et al., (Zhang, Shan et al. 2004). Human Hsp70 primers were from proprietary sequences, and all control non-targeting primer sequences were also synthesized by Dharmacon Research Inc.

First, 200 μl of the 2′-deprotection buffer was added to each 2′-ACE protected, single-stranded complementary RNA strand which was then combined, vortexed and centrifuged. The combined RNA was then incubated at 60° C. for 45 minutes in a dry-heat block. The complexes were then briefly centrifuged for 1-2 seconds and cooled at room temperature for 30 minutes to allow the RNA duplexes to anneal.

Following annealing of the duplexes, 40 μl of the 10M ammonium acetate and 1.5 ml of 100% ethanol was added to 400 μl of siRNA duplex solution and vortexed. The solution was placed at −20° C. for >16 hours or at −70° C. for 2 hours. Following freezing, the solution was centrifuged at 14000 g for 30 minutes at 4° C., then the supernatant was carefully pipetted away from the pellet. The pellet was then rinsed with 200 μl of cold 95% ethanol. The sample was finally dried under vacuum and then re-suspended in 1×siRNA Universal buffer and stored in small aliquots at −20° C. until used.

Transfection was optimized using a standard siControl Tox protocol. All transfection experiments included non-targeted siRNA.

Stock solutions of 2 μM siRNA were removed from a −20° C. freezer 30 minutes before transfection experiments. For triplicate transfections in 96-well plate format, the following master mix of reagents was prepared in RNase-free tubes for distribution of 100 μl per well:

Tube 1-17.5 μl of 2 μM siRNA was added to 17.5 μl OptiMEM media. Total volume 35 μl.

Tube 2-4.8 μl Lipofectamine was added to 30.2 μl OptiMEM media. Total volume 35 μl.

The contents of each tube were mixed and incubated at room temperature for 20 minutes. These tubes were then combined, mixed by pipetting and further incubated for 30 minutes at room temperature. Following incubation, 280 μl OptiMEM media was added to the combined solution.

### Forward Transfection Protocol: 2 Day Method

Cells were trypsinized and plated into 12- or 96-well plates at cell density of 2×10⁵, then incubated for 24 hours until adherent. Complexed siRNA and transfection agent at 100 nm was added directly to each experimental well. Plates were incubated at 37° C. for 32.5 hours for mRNA gene analysis experiments or 72 hours for protein analysis by Western Blot.

### Reverse Transfection Protocol: 1 Day Method

Complexed siRNA and transfection agent was added to each well of either 12- or 96-well plates. Cells were then trypsinized and added directly into each test well at cell density of 2×10⁵. Plates were incubated at 37° C. for 32.5 hours for mRNA gene analysis experiments or 72 hours for protein analysis by Western Blot.

Statistics

GraphPad Prism™ 4.0 running under Microsoft Windows XP®. All calculations n=6. For single variable comparisons, Student's t-test was used. For multiple variable comparisons, data were analysed by one-way ANOVA with Dunnett test performed post-hoc, where data was compared to control data; p<0.05 (95% confidence interval) or p<0.01 (99% confidence interval) was considered significant.

Results

The effects of homocysteine and H₂O₂ on endothelial cell viability were assessed first. SK-HEP-1 cells were exposed to increasing concentrations of homocysteine or H₂O₂ (range 0-50 μM) for two hours. Cell viability was determined by MTS® assay. Values are shown in FIG. 1 for the means ±SE of six independent samples. FIG. 1 illustrates the effect of homocysteine and H₂O₂ on endothelial cell viability. SK-HEP-1 cells were exposed to increasing concentrations of homocysteine or H₂O₂ (0-50 μM) for 2 hours. Cell viability was determined by MTS® assay. Values shown are the means ±SE of 6 independent samples. The results showed that concentrations of 3 μm or higher of H₂O₂ were sufficient to kill cultured endothelial cells (P<0.001). Greater than 90% (<10% survival) death was achieved by 12.5 μM H₂O₂. Concentrations of 5 μM and higher of homocysteine killed cells (p<0.001), and greater than 90% death was achieved by 25 μM homocysteine (FIG. 1).

TABLE 7
n = 6

| Concentration | | | | | | |
|---|---|---|---|---|---|---|
| μM | Homocysteine | Homocysteine | Homocysteine | Homocysteine | Homocysteine | Homocysteine |
| 50 | 0.284276 | −0.43099 | 0.944523 | 1.439709 | 3.255389 | 0.284276 |
| 25 | 1.494729 | −1.31132 | −0.32095 | −0.48601 | 0.504359 | 0.119214 |
| 12.5 | 4.906009 | −0.21091 | 0.394317 | 5.181112 | 3.860618 | 17.12059 |
| 6.25 | 62.01742 | 28.39982 | 38.2485 | 48.6474 | 67.62952 | 55.0298 |
| 3.125 | 95.96514 | 91.61852 | 82.81522 | 78.30353 | 80.94451 | 91.78358 |
| 1.562 | 100.5319 | 102.4026 | 85.18111 | 81.32966 | 81.60476 | 98.82622 |
| 0.781 | 99.76157 | 98.11095 | 102.5676 | 102.4576 | 98.71617 | 98.38605 |

| Concentration | | | | | | |
|---|---|---|---|---|---|---|
| μM | H₂O₂ | H₂O₂ | H₂O₂ | H₂O₂ | H₂O₂ | H₂O₂ |
| 50 | −0.02255 | 0.428443 | −0.24805 | 0.067649 | −0.69904 | 0.473542 |
| 25 | 3.179495 | 3.630486 | 3.630486 | 3.224594 | 3.269693 | 3.585387 |
| 12.5 | 12.42483 | 11.25225 | 10.07967 | 11.83854 | 11.74834 | 12.37973 |
| 6.25 | 32.58418 | 33.89206 | 32.31359 | 35.19994 | 32.53908 | 30.46452 |
| 3.125 | 46.88063 | 52.02194 | 50.62387 | 52.38274 | 82.7345 | 84.53848 |
| 1.562 | 96.44466 | 96.08387 | 96.44466 | 96.67016 | 98.97022 | 85.98165 |
| 0.781 | 99.42121 | 102.4429 | 98.06824 | 98.74472 | 102.3076 | 99.01532 |

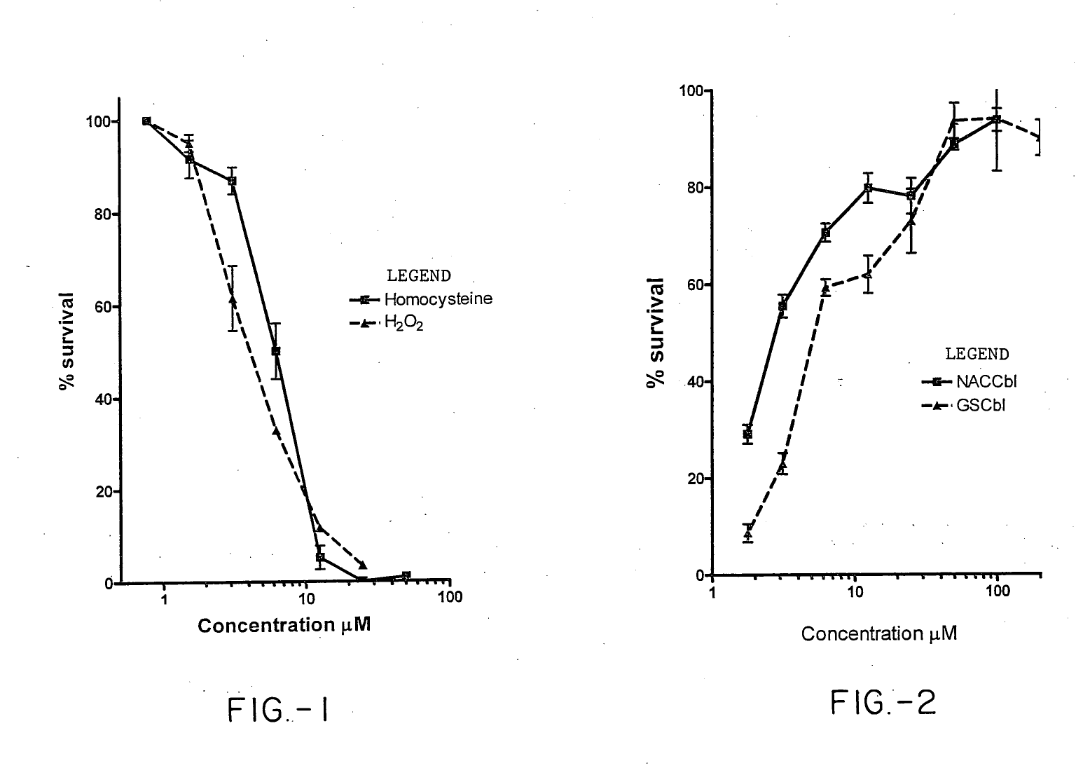

The effect of increasing concentrations of NACCbl and GSCbl to protect endothelial cells from the effects of homocysteine was assessed. SK-HEP-1 cells were exposed to increasing concentrations of NACCbl or GSCbl for two (2) hours prior to exposure to 30 μM homocysteine for 24 hours. Cell viability was determined by MTS® assay. Values shown in FIG. 2 are the means ±SE of six (6) independent samples. Results showed that pre-incubation with ≧2.0 μM of NACCbl protected cells from homocysteine-induced cell death. FIG. 2 illustrates a that NACCbl and GSCbl protect endothelial cells from the effect of homocysteine. SK-HEP-1 cells were exposed to increasing concentrations of NACCbl (▪) or GSCbl (▴) for 2 hours prior to exposure to 30 μm homocysteine for 24 hours. Cell viability was determined by MTS® assay. Values shown are the means ±SE of 6 independent samples. The level of protection increased with increasing NACCbl and survival was 80% at ≧12.5 μM NACCbl. GSCbl was also effective in protecting cells, but required a higher concentration (>80% survival protection required 50 μM GSCbl). At concentrations below 12.5 μM, the protection provided by NACCbl was significantly greater than that provided by GSCbl (p<0.001).

TABLE 8*

| Concentration | | | | | | |
|---|---|---|---|---|---|---|
| NACCbl | NACCbl | NACCbl | NACCbl | NACCbl | NACCbl | NACCbl |
| 200 | 99.44568 | 100.0554 | 105.0998 | 99.55655 | 96.11974 | 99.8337 |
| 100 | 86.86253 | 88.13747 | 99.61198 | 100.0554 | 96.72949 | 91.18626 |
| 50 | 87.47228 | 89.80045 | 86.6408 | 85.25499 | 90.63194 | 93.12639 |
| 25 | 69.56763 | 79.93348 | 65.2439 | 87.08426 | 79.60089 | 87.41685 |
| 12.5 | 79.93348 | 79.93348 | 70.78714 | 78.10421 | 76.38582 | 93.45898 |
| 6.25 | 63.41463 | 75.55432 | 68.62527 | 72.56098 | 74.72284 | 68.56985 |
| 3.125 | 44.95566 | 55.93126 | 59.70067 | 60.25499 | 53.49224 | 58.75832 |
| 1.78 | 26.44124 | 20.898 | 32.03991 | 31.54102 | 33.75832 | 30.09978 |
| 0 | 131.153 | 111.918 | 108.7583 | 87.41685 | 95.34369 | 93.84702 |
| Concentration | | | | | | |
| GSCbl | GSCbl | GSCbl | GSCbl | GSCbl | GSCbl | GSCbl |
| 200 | 81.87362 | 76.38582 | 99.6674 | 96.72949 | 92.23947 | 93.18182 |
| 100 | 51.99557 | 87.86031 | 89.91132 | 93.51441 | 108.5366 | 132.2062 |
| 50 | 81.3193 | 103.6585 | 84.31264 | 94.62307 | 102.2727 | 95.50999 |
| 25 | 60.36586 | 65.13304 | 75.77605 | 62.74945 | 69.73393 | 104.102 |
| 12.5 | 56.76275 | 71.50776 | 60.47672 | 56.153 | 51.49668 | 75.49889 |
| 6.25 | 60.42128 | 62.02883 | 64.41241 | 52.32816 | 58.64745 | 57.76053 |
| 3.125 | 30.76497 | 14.74501 | 25.44346 | 23.61419 | 20.898 | 22.56098 |
| 1.78 | 14.13525 | 9.811529 | 6.485587 | 0.997783 | 11.58537 | 9.09091 |
| 0 | 132.2062 | 51.99557 | 87.86031 | 89.91132 | 93.51441 | 108.5366 |

*Varying Concentrations of NACCbl and GSCbl, followed by exposure to 30 μM homocysteine for 24 hours.

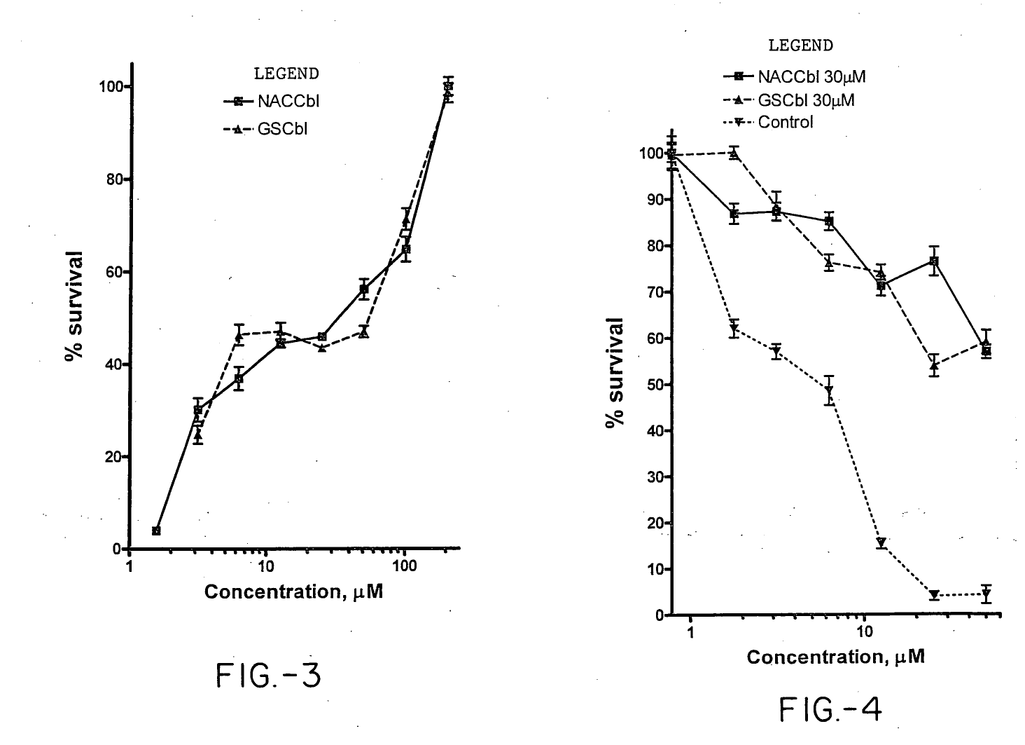

The effect of increasing concentrations of NACCbl and GSCbl to protect endothelial cells from the effect of H₂O₂ was assessed. SK-HEP-1 cells were exposed to increasing concentrations of NACCbl or GSCbl for two (2) hours prior to exposure to 25 μM H₂O₂ for 24 hours. Cell viability was determined by MTS® assay. Values shown in FIG. 3 are the means ±SE of 6 independent samples. FIG. 3 illustrates that NACCbl and GSCbl protect endothelial cells from the effect of H₂O₂. SK-HEP-1 cells were exposed to increasing concentrations of NACCbl (▪) or GSCbl (▴) for 2 hours prior to exposure to 25 μM H₂O₂ for 24 hours. Cell viability was determined by MTS® assay. Values shown are the means ±SE of 6 independent samples. The results show that preincubation with ≧2.0 μM NACCbl protected cells from H₂O₂-induced cell death. There was no difference between the protection afforded by NACCbl and that by GSCbl. Both required 100 μM to achieve >80% survival.

TABLE 9*
n = 6

| Concentration | | | | | | |
|---|---|---|---|---|---|---|
| NACCbl | NACCbl | NACCbl | NACCbl | NACCbl | NACCbl | NACCbl |
| 200 | 100.7246 | 99.7365 | 101.8445 | 107.3123 | 92.68775 | 97.56258 |
| 100 | 61.5942 | 56.58762 | 70.22398 | 74.37418 | 64.22925 | 61.79184 |
| 50 | 56.65349 | 49.93412 | 60.2108 | 63.24111 | 57.11463 | 49.93412 |
| 25 | 46.90382 | 46.77207 | 45.25692 | 42.68774 | 45.71805 | 48.15547 |
| 12.5 | 45.25692 | 41.69961 | 44.99342 | 45.5863 | 42.22661 | 47.36496 |
| 6.25 | 42.8195 | 42.09486 | 39.39394 | 30.89592 | 27.99737 | 38.4058 |
| 3.125 | 26.8116 | 19.49934 | 35.83663 | 30.50066 | 35.24374 | 33.00395 |
| 1.562 | 4.677207 | 1.844532 | 5.270093 | 1.646904 | 4.347825 | 5.599474 |
| 0 | 128.4585 | 124.1107 | 134.3215 | 115.8103 | 128.4585 | 108.3663 |

| Concentration | | | | | | |
|---|---|---|---|---|---|---|
| GSCbl | GSCbl | GSCbl | GSCbl | GSCbl | GSCbl | GSCbl |
| 200 | 94.07115 | 100.0659 | 102.8327 | 93.87352 | 105.5995 | 94.72991 |
| 100 | 64.62451 | 64.62451 | 77.86562 | 71.60738 | 72.59553 | 76.5481 |
| 50 | 42.68774 | 45.71805 | 48.15547 | 46.90382 | 46.77207 | 51.84454 |
| 25 | 41.23847 | 45.5863 | 42.22661 | 45.25692 | 41.69961 | 44.99342 |
| 12.5 | 44.26878 | 54.34783 | 50.52701 | 47.03558 | 44.13702 | 42.09486 |
| 6.25 | 48.68248 | 56.25824 | 45.98156 | 43.34651 | 42.09486 | 41.96311 |
| 3.125 | 20.88274 | 30.89592 | 21.34387 | 26.8116 | 19.49934 | 29.24901 |
| 1.562 | 6.060607 | 1.119894 | −1.58103 | 2.635046 | −0.46113 | −7.83926 |
| 0 | 117.3254 | 129.9737 | 133.5968 | 122.4638 | 120.4875 | 115.1515 |

*Variable concentrations of NACCbl and GSCbl, followed by exposure to 25 μM H₂O₂ for 24 hours.

The effect of a constant concentration of NACCbl and GSCbl to protect cells against variable concentrations of homocysteine was assessed. Cells were pre-treated with NACCbl or GSCbl (30 μM) for two hours and then exposed to variable concentrations of homocysteine for a further two hours. Cell viability was determined by MTS® assay. Data shown in FIG. 4 are representative of means ±SE of 6 independent samples. FIG. 4 illustrates that NACCbl (▪) and GSCbl (▴) protect endothelial cells from the effect of homocysteine. Cells were pre-treated with NACCbl or GSCbl (30 μM) for two hours and then exposed to variable concentrations of homocysteine for a further two hours. Cell viability was determined by MTS® assay. Data shown are representative of means ±SE of 6 independent samples. The results show that pre-incubation of cells with 30 μM NACCbl or GSCbl protected cells from homocysteine-induced cell death. There was a decrease in protection as homocysteine concentration increased, but there was still ˜60% survival at 50 μM homocysteine.

TABLE 10*

| Variable | | | | | | |
|---|---|---|---|---|---|---|
| Concentration | NACCbl | NACCbl | NACCbl | NACCbl | NACCbl | NACCbl |
| Hcy | 30 μM | 30 μM | 30 μM | 30 μM | 30 μM | 30 μM |
| 200 | −8.4058 | −18.6473 | 11.11111 | 12.75363 | 4.444445 | −1.25604 |
| 100 | 43.28503 | 33.42996 | 40.96619 | 44.92754 | 37.77778 | 41.15942 |
| 50 | 61.5459 | 59.22706 | 55.94203 | 57.68116 | 56.61837 | 50.5314 |
| 25 | 71.0145 | 77.68117 | 86.47344 | 83.96136 | 73.81644 | 66.2802 |
| 12.5 | 66.57005 | 67.24638 | 77.48792 | 72.36715 | 77.00484 | 66.37682 |
| 6.25 | 89.46861 | 80.86957 | 90.62803 | 78.4541 | 85.70049 | 86.18359 |
| 3.125 | 94.87923 | 85.89373 | 87.24638 | 81.35267 | 90.24156 | 84.05798 |
| 1.78 | 88.40581 | 92.85025 | 90.72464 | 88.21256 | 79.03381 | 81.64253 |
| 0.781 | 105.314 | 107.7295 | 97.2947 | 93.33334 | 86.85991 | 109.4686 |

| Variable | | | | | | |
|---|---|---|---|---|---|---|
| Concentration | GSCbl | GSCbl | GSCbl | GSCbl | GSCbl | GSCbl |
| Hcy | 30 μM | 30 μM | 30 μM | 30 μM | 30 μM | 30 μM |
| 200 | 5.794027 | 13.84914 | −4.27487 | 6.535942 | −5.86469 | −16.0396 |
| 100 | 8.019773 | 20.95035 | 15.43896 | 28.05157 | 23.2821 | 33.03303 |
| 50 | 66.52534 | 53.91274 | 66.41935 | 56.35047 | 59.6361 | 51.47501 |
| 25 | 64.29959 | 52.85284 | 53.38278 | 46.70552 | 51.26302 | 54.76063 |
| 12.5 | 76.38226 | 66.73732 | 72.56669 | 77.12418 | 75.42837 | 76.80621 |
| 6.25 | 76.38226 | 76.80621 | 77.33616 | 79.03197 | 67.9032 | 80.19784 |
| 3.125 | 88.35894 | 76.9122 | 97.8979 | 89.52482 | 83.58947 | 94.71825 |
| 1.78 | 104.3632 | 94.2943 | 99.6997 | 101.5015 | 100.8656 | 99.27575 |
| 0.781 | 111.4644 | 100.9716 | 92.91645 | 97.47395 | 93.3404 | 100.9716 |

| Variable | | | | | | |
|---|---|---|---|---|---|---|
| Concentration | | | | | | |
| Hcy | Control | Control | Control | Control | Control | Control |
| 200 | −3.784322 | 0.825153 | −0.625979 | 2.361645 | 4.666382 | 3.300612 |
| 100 | 3.385972 | −3.784322 | −5.150093 | 6.032153 | 0.44103 | −0.924741 |
| 50 | 7.09916 | 4.666382 | −3.912363 | 7.09916 | 1.508038 | 8.934416 |
| 25 | 0.825153 | 3.215251 | 3.727415 | 7.568644 | 5.391947 | 2.745767 |
| 12.5 | 10.55627 | 14.90966 | 17.47048 | 15.16574 | 16.27543 | 17.47048 |
| 6.25 | 43.50548 | 62.37018 | 50.80381 | 43.2494 | 41.75558 | 49.69412 |
| 3.125 | 54.517 | 58.35823 | 58.40091 | 50.88917 | 63.01038 | 56.77906 |
| 1.78 | 63.05307 | 55.37061 | 67.5345 | 59.63864 | 67.1077 | 59.42524 |
| 0.781 | 100.441 | 102.2336 | 107.782 | 95.44743 | 98.5631 | 95.53279 |

*Constant concentrations of NACCbl and GSCbl (30 μM), followed by variable concentrations of homocysteine for a further two hours.

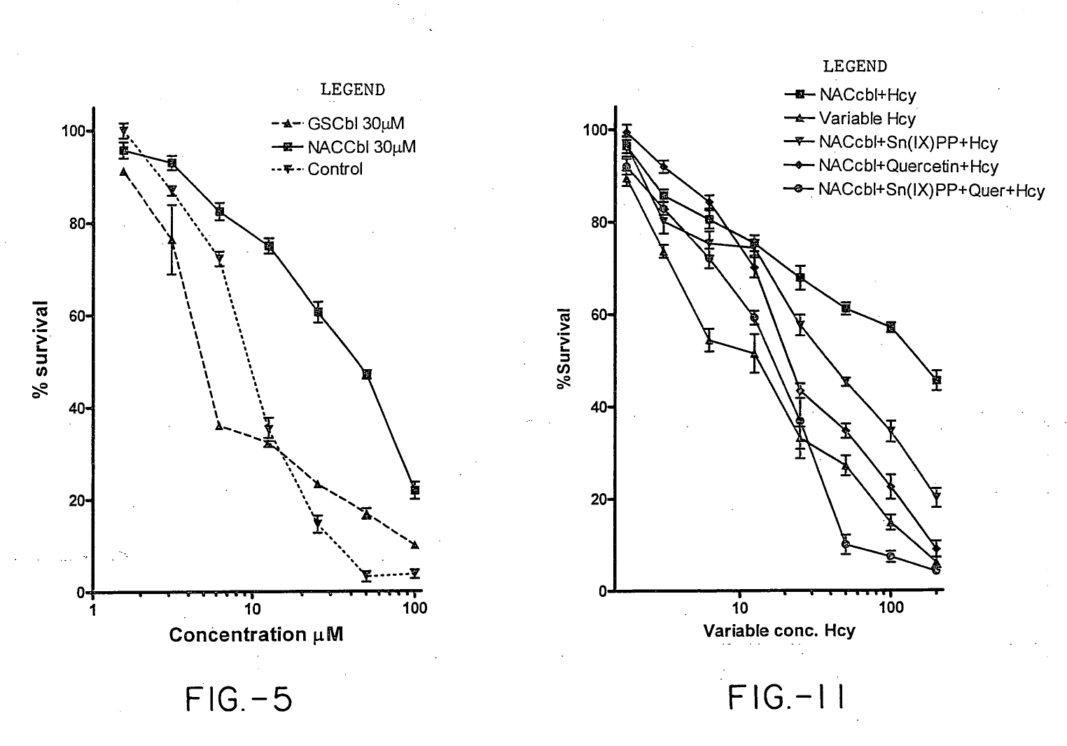

Protection of endothelial cells by NACCbl (at a constant concentration of 30 μM) was also observed when cells were exposed to variable concentrations of H₂O₂, however, the protection decreased below 60% survival above 25 μM H₂O₂ (FIG. 5). FIG. 5 illustrates that NACCbl (▪) and GSCbl (▴) protect endothelial cells from the effect of H₂O₂. Cells were pre-treated with NACCbl or GSCbl (30 μM) for two hours and then exposed to variable concentrations of H₂O₂ for a further two hours. Cell viability was determined by MTS® assay. Data shown are representative of means ±SE of 6 independent samples. GSCbl (at a constant concentration of 30 μM) did not provide significant protection above 7.5 μM H₂O₂ (also FIG. 5).

TABLE 11*
n = 6

| H₂O₂ | | | | | | |
|---|---|---|---|---|---|---|
| Concentration | NACCbl | NACCbl | NACCbl | NACCbl | NACCbl | NACCbl |
| μM | 30 μM | 30 μM | 30 μM | 30 μM | 30 μM | 30 μM |
| 200 | 5.2108 | −0.61582 | 7.153008 | −1.65798 | 1.989578 | 0.663193 |
| 100 | 29.98579 | 23.96968 | 19.56419 | 18.901 | 17.52724 | 20.89057 |
| 50 | 50.21317 | 48.27095 | 44.955 | 45.71293 | 48.17622 | 44.57603 |
| 25 | 67.12459 | 66.74561 | 61.86642 | 56.18191 | 55.3766 | 55.94505 |
| 12.5 | 77.16722 | 76.97774 | 76.59877 | 67.12459 | 77.6883 | 74.13548 |
| 6.25 | 88.20464 | 87.58881 | 82.14117 | 81.57272 | 78.06727 | 77.11985 |
| 3.125 | 88.20464 | 93.17859 | 92.89436 | 99.76315 | 92.42065 | 91.80482 |
| 1.56 | 98.53151 | 91.37849 | 98.72099 | 100.1421 | 89.76788 | 96.11559 |
| 0.781 | 93.46281 | 102.7949 | 99.24207 | 101.3738 | 103.2686 | 95.45239 |
| 0 | 102.9844 | 100.8053 | 103.2212 | 107.9583 | 101.8948 | 98.43676 |

| H₂O₂ | | | | | | |
|---|---|---|---|---|---|---|
| Concentration | GSCbl | GSCbl | GSCbl | GSCbl | GSCbl | GSCbl |
| μM | 30 μM | 30 μM | 30 μM | 30 μM | 30 μM | 30 μM |
| 200 | 1.326385 | 7.153008 | 3.60019 | 1.942207 | 5.921364 | 0.757935 |
| 100 | 10.80057 | 12.36381 | 11.5585 | 9.995263 | 6.679299 | 8.526765 |
| 50 | 15.01658 | 20.8432 | 18.94837 | 13.12174 | 15.6324 | 17.62198 |
| 25 | 23.54334 | 26.52771 | 22.92752 | 24.44339 | 22.02748 | 20.41686 |
| 12.5 | 29.7963 | 32.02274 | 36.09664 | 32.40171 | 29.98579 | 33.91758 |
| 6.25 | 36.52297 | 39.7442 | 34.48603 | 34.72288 | 34.86499 | 36.09664 |
| 3.125 | 40.31265 | 91.33112 | 87.3046 | 82.33065 | 79.77262 | 77.59356 |
| 1.56 | 93.55756 | 90.81004 | 92.32591 | 88.25201 | 90.81004 | 91.8522 |
| 0.781 | 96.11559 | 98.38939 | 97.3946 | 98.0578 | 96.02085 | 95.31028 |
| 0 | 99.24207 | 102.2264 | 105.5898 | 103.6002 | 104.0265 | 104.5476 |

| H₂O₂ | | | | | | |
|---|---|---|---|---|---|---|
| Concentration | | | | | | |
| μM | Control | Control | Control | Control | Control | Control |
| 200 | 4.263382 | −1.61061 | −0.52108 | −2.17906 | 1.56324 | −1.51587 |
| 100 | 1.326385 | 0.757935 | 4.547607 | 3.458076 | 5.447654 | 6.679299 |
| 50 | 3.837044 | 7.153008 | −1.56324 | 3.60019 | 3.837044 | 2.321175 |
| 25 | 19.42208 | 10.27949 | 13.73757 | 19.75367 | 15.58503 | 8.810989 |
| 12.5 | 34.86499 | 40.31265 | 44.71814 | 30.22264 | 32.49645 | 28.61203 |
| 6.25 | 72.38276 | 73.04596 | 67.17196 | 72.28801 | 78.63572 | 69.58788 |
| 3.125 | 87.92043 | 87.54145 | 83.98862 | 90.81004 | 84.41497 | 87.25723 |
| 1.56 | 93.46281 | 98.29465 | 99.33681 | 101.0422 | 104.6897 | 103.2212 |
| 0.781 | 93.46281 | 102.3212 | 96.11559 | 100.8053 | 103.2686 | 101.5632 |
| 0 | 110.2795 | 108.8584 | 109.9479 | 113.6902 | 109.9479 | 114.35354 |

*Constant concentrations of NACCbl at 30 μM, followed by exposure to variable concentrations of H₂O₂.

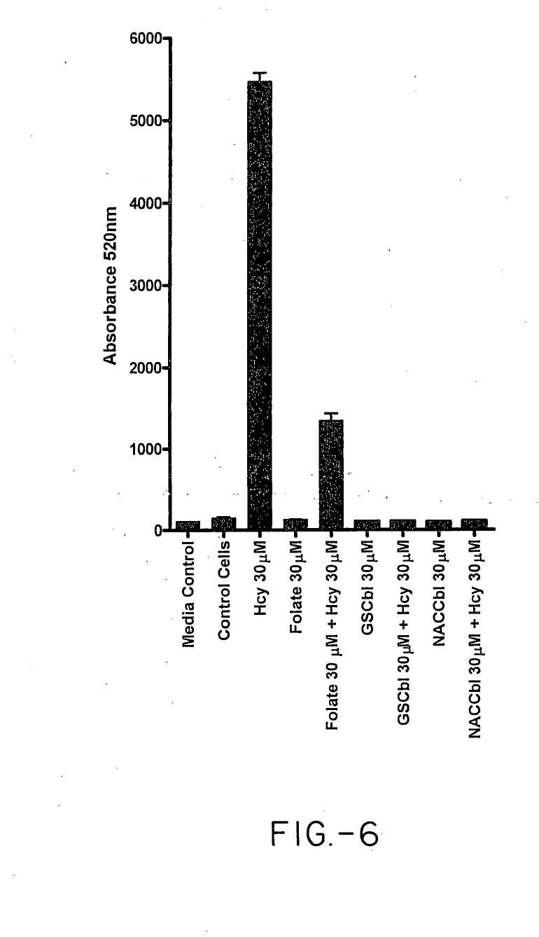

The effects of NACCbl, GSCbl and folate to protect endothelial cells from apoptosis induced by homocysteine were also assessed. Homocysteine-induced apoptosis in cells was measured by Caspase-3 activity (FIG. 6). FIG. 6 illustrates that NACCbl, GSCbl and folate protect endothelial cells from apoptosis induced by homocysteine. Cells were pre-treated with NACCbl, GSCbl or folate for two hours and then exposed to 0 or 30 μM homocysteine for a further two hours. Cell viability was determined by MTS® assay. Data shown are representative of means ±SE of 6 independent samples. Cells were pretreated with NACCbl, GSCbl or folate (all at 30 μM) for two hours and then exposed to 0 or 30 μM homocysteine for a further two hours. Cell viability was determined by MTS® assay. Data shown in FIG. 6 are representative of means ±SE of 6 independent samples. The results showed that pre-incubation with folate provided partial protection against apoptosis induced by homocysteine, whereas both NACCbl and GSCbl provided total protection.

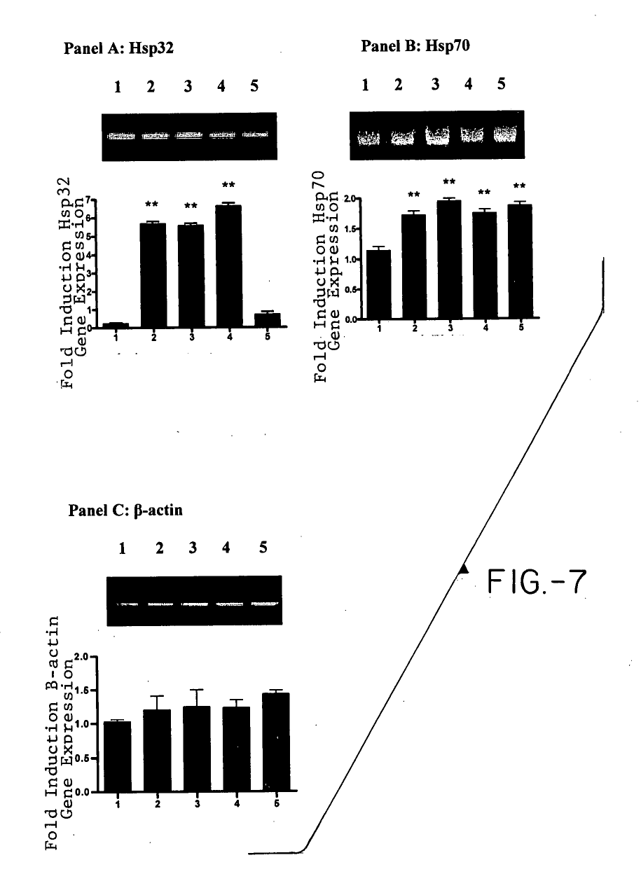

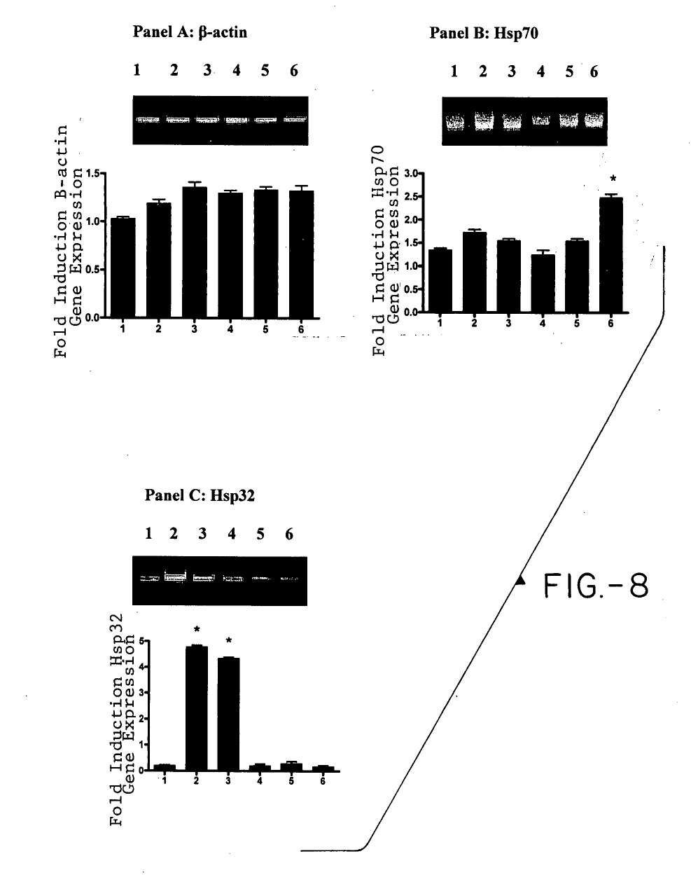

Efforts were made to elicit the mechanism by which NACCbl affords protection to endothelial cells. Experiments were conducted using Hsp32 and Hsp 70 gene expression as the basis for study. Homocysteine is an oxidative stress inducer and as such should induce the expression of heat shock protein (Hsp) Hsp32. FIG. 7 shows the results of oxidative stress on Hsp32 and Hsp 70 gene expression with no treatment, Hcy 30 μM, folate 30 μM, folate 30 μM plus Hcy 30 μM, and heat shock at 42° C. (all treatments for 2 hours). The densitometric data are presented in FIG. 6 as means ±SEM of n=3 separate experiments (p<0.05 or p<0.01, treatment vs. control). The results showed that the Hsp70 gene was expressed in control cells (no treatment), whereas the Hsp32 gene was not (FIG. 7). FIG. 7 illustrates that the effect of oxidative stress on Hsp32 and Hsp70 gene expression in SK-HEP-1 Cells. All panels: 1, no treatment; 2: Hcy 30 μM, 2 hr; 3: folate 30 μM, 2 hr; 4: folate 30 μM, 2 hr+Hcy 30 μM, 2 hr; 5: Heat shock 42° C., 2 hr. The densitometric data are presented as means ±SEM of n=3 separate experiments. *p<0.05 or **p<0.01, treatment vs. control. An increase in expression of both genes was induced by 30 μM homocysteine, by 30 μM folate and by folate plus homocysteine (both at 30 μM concentration). A 42° C. heat shock increased expression of Hsp70, but not Hsp32, confirming that Hsp32 is specifically induced by oxidative stress. FIG. 8 illustrates a Gene Expression Study: Oxidative stress of SK-HEP-1 Cells. Panel A, B and C: Lane 1 shows the control, no treatment; lane 2: H₂O₂ 25 μM, 2 hr; lane 3: folate 30 μM, 2 hr+H₂O₂ 25 μM, 1 hr; lane 4: Sn(IX) PP 25 μM, 2 hr; lane 5: Sn(IX) PP 25 μM, 2 hr+folate 30 μM, 2 hr+H₂O₂ 25 μM, 1 hr; lane 6: Heat shock 42° C., 2 hr. Panel B shows fold induction difference in β-actin gene expression from Control=1.

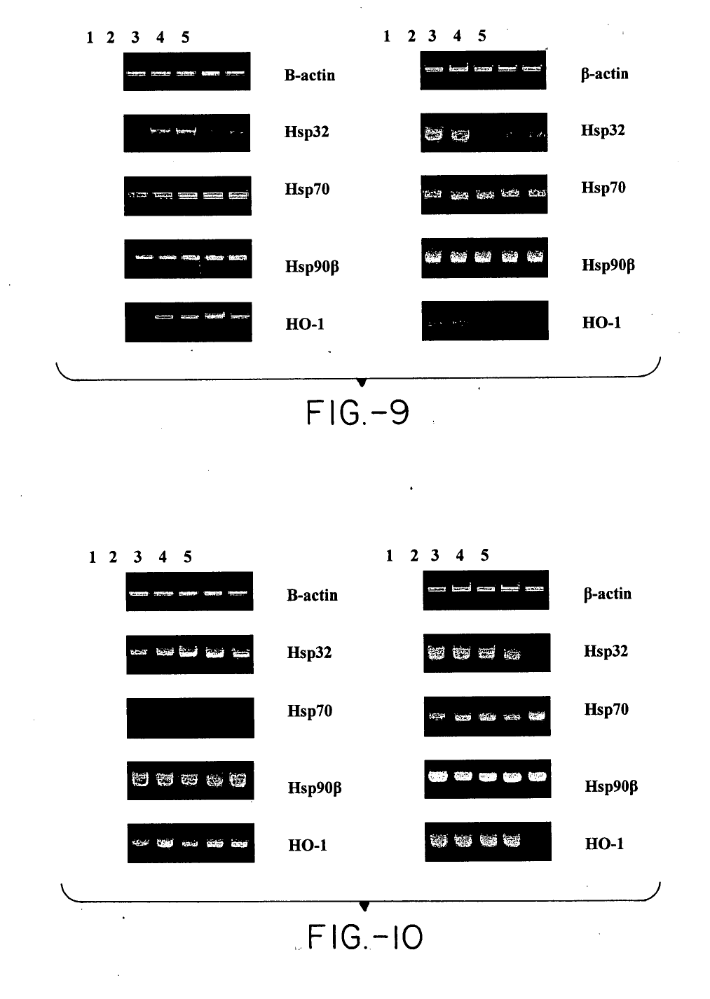

FIG. 9 illustrates that Hsp32 gene expression is induced by homocysteine and NACCbl, and can be inhibited by Sn(IX) protoporphyrin. SK-HEP-1 cells were treated with various compounds prior to PCR. (note HO1=Hsp32). FIG. 9 also shows that the Hsp32 gene can be inhibited by Sn (IX) protoporphyrin (25 μM). Other molecules, including NACCbl and GSCbl, that protect against homocysteine-induced cell death also induce Hsp32 (FIGS. 7, 9 and 10). Quercitin 15 μM inhibits Hsp70 gene expression, but does not inhibit Hsp32 gene expression (FIGS. 9 and 10).

Two alternative approaches were used to determine whether Hsp32 or Hsp70 have a role in the mechanism by which NACCbl protects against homocysteine induced cell death. Hsp32 was inhibited using either Sn (IX) protoporhyrin or using an siRNA which specifically knocks out Hsp32. Hsp70 was inhibited using quercitin or using an siRNA which specifically knocks out Hsp70. In addition, two methods were used to inhibit both Hsp32 and Hsp70: chemically Sn (IX) protoporphyrin plus quercitin was used; and then, directly, a combination of the siRNA for both genes was used simultaneously.

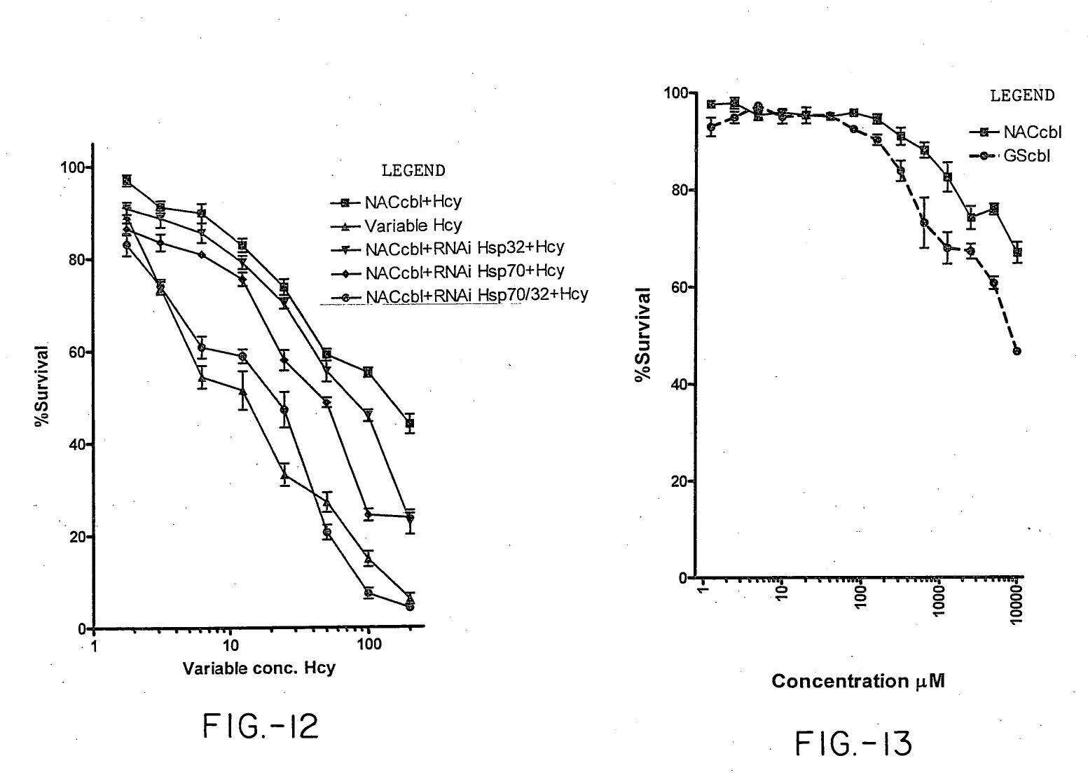

In one set of experiments, cells were pre-treated with NACCbl (30 μM) for two hours in the presence or absence of Sn (IX) protoporphyrin or quercitin and then exposed to variable concentrations of homocysteine for a further two hours. Cell viability was determined by MTS® assay. Data shown in FIG. 11 are representative of means ±SE of 6 independent samples. In another set of experiments, cells were pre-treated NACCbl (30 μM) for two (2) hours in the presence or absence of siRNA specific for Hsp32 or Hsp70 and then exposed to variable concentrations of homocysteine for a further two hours. FIG. 11 illustrates that NACCbl protects endothelial cells from the effect of homocysteine via a mechanism involving Hsp70 and Hsp32. Cells were pre-treated with NACCbl (30 μM) for two hours in the presence or absence of Sn(IX) protoporphyrin or quercitin and then exposed to variable concentrations of homocysteine for a further two hours. Cell viability was determined by MTS® assay. Data shown are representative of means ±SE of 6 independent samples. FIG. 12 illustrates NACCbl protects endothelial cells from the effect of homocysteine. Cells were pre-treated NACCbl (30 μM) for two hours in the presence or absence of siRNA specific for Hsp70 and then exposed to variable concentrations of homocysteine for a further two hours. Cell viability was determined by MTS® assay. Data shown are representative of means ±SE of 6 independent samples. Cell viability was determined by MTS® assay. Data shown are representative of means ±SE of 6 independent samples. The results showed that inhibition of Hsp32 by both methods (chemically and directly) reduced the protection by NACCbl by 10-20% (p<0.001, FIGS. 11 and 12). Inhibition of Hsp70 by both methods (chemically and directly) reduced the protection by NACCbl by 20-40% (p<0.001, FIGS. 11 and 12). Inhibition of both Hsp32 and Hsp70 together by both methods (chemically and directly) totally removed protection by NACCbl (FIGS. 11 and 12).

TABLE 12
(FIG. 11)

| Variable | NACCbl + Hcy (N = 6) | | | | | |
|---|---|---|---|---|---|---|
| Conc. of Hcy | 1 | 2 | 3 | 4 | 5 | 6 |
| 200 | 1.378 | 1.167 | 1.33 | 1.456 | 1.491 | 1.537 |
| 100 | 1.678 | 1.592 | 1.694 | 1.773 | 1.732 | 1.639 |
| 50 | 1.678 | 1.832 | 1.881 | 1.73 | 1.74 | 1.855 |
| 25 | 2.1 | 2.19 | 1.879 | 1.937 | 1.779 | 1.837 |
| 12.5 | 2.21 | 2.11 | 2.045 | 2.065 | 2.101 | 2.324 |
| 6.25 | 2.41 | 2.4 | 2.322 | 2.201 | 2.115 | 2.187 |
| 3.125 | 2.453 | 2.338 | 2.36 | 2.317 | 2.418 | 2.535 |
| 1.78 | 2.647 | 2.551 | 2.789 | 2.89 | 2.549 | 2.611 |
| 0 | 2.885 | 2.698 | 2.821 | 2.771 | 2.691 | 2.7 |
| Variable | Variable Hcy (n = 6) | | | | | |
| Conc. of Hcy | 1 | 2 | 3 | 4 | 5 | 6 |
| 200 | 0.33 | 0.312 | 0.39 | 0.302 | 0.421 | 0.406 |
| 100 | 0.654 | 0.449 | 0.466 | 0.501 | 0.522 | 0.51 |
| 50 | 0.678 | 0.881 | 0.602 | 0.77 | 0.724 | 0.779 |
| 25 | 0.692 | 0.956 | 0.876 | 0.882 | 0.937 | 0.74 |
| 12.5 | 1.104 | 1.001 | 1.042 | 1.114 | 1.324 | 1.479 |
| 6.25 | 1.304 | 1.406 | 1.227 | 1.176 | 1.15 | 1.119 |
| 3.125 | 1.543 | 1.487 | 1.656 | 1.63 | 1.593 | 1.558 |
| 1.78 | 1.746 | 1.932 | 1.95 | 1.83 | 1.837 | 1.884 |
| 0 | 2.109 | 2.11 | 1.999 | 2.056 | 2.1 | 1.936 |

| Variable | NACCbl + Sn(IX)PP + Hcy (n = 6) | | | | | |
|---|---|---|---|---|---|---|
| Conc. of Hcy | 1 | 2 | 3 | 4 | 5 | 6 |
| 200 | 0.679 | 0.837 | 0.882 | 0.91 | 0.723 | 0.577 |
| 100 | 1.29 | 1.117 | 1.104 | 1.002 | 0.994 | 1.336 |
| 50 | 1.488 | 1.47 | 1.43 | 1.379 | 1.337 | 1.402 |
| 25 | 1.876 | 1.749 | 1.678 | 1.63 | 1.557 | 1.921 |
| 12.5 | 2.123 | 2.301 | 2.244 | 2.221 | 2.109 | 2.008 |
| 6.25 | 2.443 | 2.331 | 2.156 | 2.004 | 2.078 | 2.14 |
| 3.125 | 2.578 | 2.432 | 2.29 | 2.278 | 2.21 | 2.11 |
| 1.78 | 2.567 | 2.69 | 2.733 | 2.779 | 2.821 | 2.879 |
| 0 | 2.6788 | 2.897 | 2.701 | 2.857 | 2.891 | 2.945 |

| Variable | NACCbl + Quercetin + Hcy (n = 6) | | | | | |
|---|---|---|---|---|---|---|
| Conc. of Hcy | 1 | 2 | 3 | 4 | 5 | 6 |
| 200 | 0.573 | 0.423 | 0.444 | 0.301 | 0.567 | 0.583 |
| 100 | 1.111 | 1.678 | 0.732 | 0.883 | 0.891 | 0.693 |
| 50 | 1.021 | 1.247 | 1.207 | 1.197 | 1.177 | 1.022 |
| 25 | 1.478 | 1.501 | 1.376 | 1.321 | 1.297 | 1.25 |
| 12.5 | 2.123 | 2.116 | 2.046 | 2.1 | 2.187 | 1.794 |
| 6.25 | 2.467 | 2.409 | 2.337 | 2.365 | 2.401 | 2.588 |
| 3.125 | 2.765 | 2.631 | 2.661 | 2.589 | 2.603 | 2.509 |
| 1.78 | 2.678 | 2.889 | 2.921 | 2.703 | 2.831 | 2.899 |
| 0 | 2.991 | 2.851 | 2.956 | 2.756 | 2.764 | 2.678 |

| Variable | NACCbl + Sn(IX)PP + Quercetin + Hcy (n = 6) | | | | | |
|---|---|---|---|---|---|---|
| Conc. of Hcy | 1 | 2 | 3 | 4 | 5 | 6 |
| 200 | 0.278 | 0.389 | 0.41 | 0.336 | 0.333 | 0.417 |
| 100 | 0.367 | 0.401 | 0.476 | 0.507 | 0.551 | 0.367 |
| 50 | 0.746 | 0.478 | 0.539 | 0.337 | 0.439 | 0.551 |
| 25 | 1.123 | 1.034 | 0.786 | 1.809 | 1.922 | 0.664 |
| 12.5 | 1.678 | 1.89 | 1.709 | 1.902 | 1.834 | 1.88 |
| 6.25 | 2.123 | 2.39 | 2.167 | 2.117 | 2.17 | 1.963 |
| 3.125 | 2.456 | 2.389 | 2.301 | 2.489 | 2.447 | 2.567 |
| 1.78 | 2.789 | 2.657 | 2.489 | 2.699 | 2.798 | 2.662 |
| 0 | 2.888 | 2.79 | 2.901 | 2.979 | 2.991 | 2.804 |

TABLE 13
(FIG. 12)

| | | |
|---|---|---|
| Variable | | |
| Conc. of | | |
| Hcy | Mean | SEM |

NACCbl + Hcy

| | | |
|---|---|---|
| 200 | 43.9595 | 2.101288 |
| 100 | 55.16888 | 1.013695 |
| 50 | 59.06556 | 1.271858 |
| 25 | 73.75504 | 1.724869 |
| 12.5 | 82.83664 | 1.460632 |
| 6.25 | 89.85452 | 2.128654 |
| 3.125 | 91.34142 | 1.305542 |
| 1.78 | 97.04544 | 1.198051 |
| 0 | 100 | 0.929276 |

Variable Hcy

| | | |
|---|---|---|
| 200 | 6.114708 | 1.68879 |
| 100 | 14.81961 | 1.644149 |
| 50 | 27.14154 | 2.160151 |
| 25 | 33.14524 | 2.430397 |
| 12.5 | 51.47086 | 4.194286 |
| 6.25 | 54.41258 | 2.442547 |
| 3.125 | 73.70027 | 1.393837 |
| 1.78 | 89.53746 | 1.702477 |
| 0 | 100 | 1.608582 |

NACCbl + RNAi + Hsp 32 + Hcy

| | | |
|---|---|---|
| 200 | 22.71906 | 2.604406 |
| 100 | 45.81881 | 1.243889 |
| 50 | 55.51684 | 2.269596 |
| 25 | 70.24131 | 1.135635 |
| 12.5 | 79.12633 | 1.428551 |
| 6.25 | 85.54007 | 2.120552 |
| 3.125 | 88.81791 | 2.042473 |
| 1.78 | 91.044 | 1.296631 |
| 0 | 100 | 2.045876 |

NACCbl + RNAi + Hsp 70/32 + Hcy

| | | |
|---|---|---|
| 200 | 4.156479 | 0.830308 |
| 100 | 7.328696 | 1.192364 |
| 50 | 20.61939 | 1.645706 |
| 25 | 47.21334 | 3.858868 |
| 12.5 | 58.88659 | 1.501209 |
| 6.25 | 60.86139 | 2.375267 |
| 3.125 | 74.12074 | 1.424911 |
| 1.78 | 83.07944 | 2.454529 |
| 0 | 100 | 1.008469 |

NACCbl + RNAi + Hsp 70 + Hcy

| | | |
|---|---|---|
| 200 | 23.74355 | 0.881615 |
| 100 | 24.32296 | 1.313255 |
| 50 | 48.75299 | 1.06419 |
| 25 | 57.9481 | 2.211338 |
| 12.5 | 75.53218 | 1.459312 |
| 6.25 | 80.8918 | 0.7901 |
| 3.125 | 83.51177 | 1.823074 |
| 1.78 | 86.53482 | 1.290047 |
| 0 | 100 | 1.468781 |

The concentrations needed for clinical treatments and supplementation may well need to be higher than those used for the cellular experiments described herein. Equally important to the use of thiolatocobalamins to protect endothelial and other cells from the effects of oxidative stress is their safety or lack of a detrimental effect on exposed cells. The effect of high concentrations of NACCbl and GSCbl on SK-HEP-1 cells was evaluated. Cells were exposed to both compounds in increasing concentrations over twenty-four (24) hours. Cell viability was determined by MTS® assay. Data shown in FIG. 13 are representative of means ±SE of 3 independent samples. FIG. 13 illustrates that the effect of high concentrations of NACCbl and GSCbl on SK-HEP-1 cells. Cells were exposed to the compounds for 24 hours and cell viability was determined by MTS® assay. Data shown are representative of means ±SE of 3 independent samples. The results showed that increasing the concentrations of NACCbl and GSCbl did not affect cell viability until reaching concentrations above 0.2 mM. Above this concentration, there was a decrease in survival but even at 10 mM over 60% survival was observed in the NACCbl treated cells (FIG. 13). Above 0.2 mM concentration, GSCbl caused greater cell death than NACCbl (FIG. 13).

TABLE 14

| Variable | | | | | | |
|---|---|---|---|---|---|---|
| Conc. | NACCbl | NACCbl | NACCbl | GSCbl | GSCbl | GSCbl |
| 10000 | 1.908 | 1.82 | 1.732 | 1.322 | 1.342 | 1.366 |
| 5000 | 2.081 | 2.003 | 2.004 | 1.728 | 1.67 | 1.623 |
| 2500 | 1.989 | 1.891 | 2.085 | 1.775 | 1.81 | 1.896 |
| 1250 | 2.2 | 2.055 | 2.3 | 1.973 | 1.846 | 1.71 |
| 625 | 2.358 | 2.242 | 2.347 | 2.197 | 1.912 | 1.787 |
| 312.5 | 2.409 | 2.301 | 2.437 | 2.306 | 2.204 | 2.138 |
| 156.25 | 2.484 | 2.42 | 2.492 | 2.407 | 2.37 | 2.318 |
| 78.125 | 2.521 | 2.488 | 2.475 | 2.444 | 2.406 | 2.402 |
| 39.062 | 2.503 | 2.474 | 2.453 | 2.492 | 2.484 | 2.468 |
| 19.531 | 2.485 | 2.466 | 2.502 | 2.553 | 2.476 | 2.42 |
| 9.765 | 2.505 | 2.474 | 2.514 | 2.532 | 2.476 | 2.42 |
| 4.882 | 2.532 | 2.463 | 2.457 | 2.543 | 2.528 | 2.519 |
| 2.441 | 2.592 | 2.536 | 2.499 | 2.527 | 2.434 | 2.46 |
| 1.22 | 2.546 | 2.524 | 2.538 | 2.347 | 2.433 | 2.503 |
| 0.61 | 2.572 | 2.498 | 2.473 | 2.554 | 2.376 | 2.53 |
| 0 | 2.61 | 2.688 | 2.634 | 2.487 | 2.441 | 2.52 |

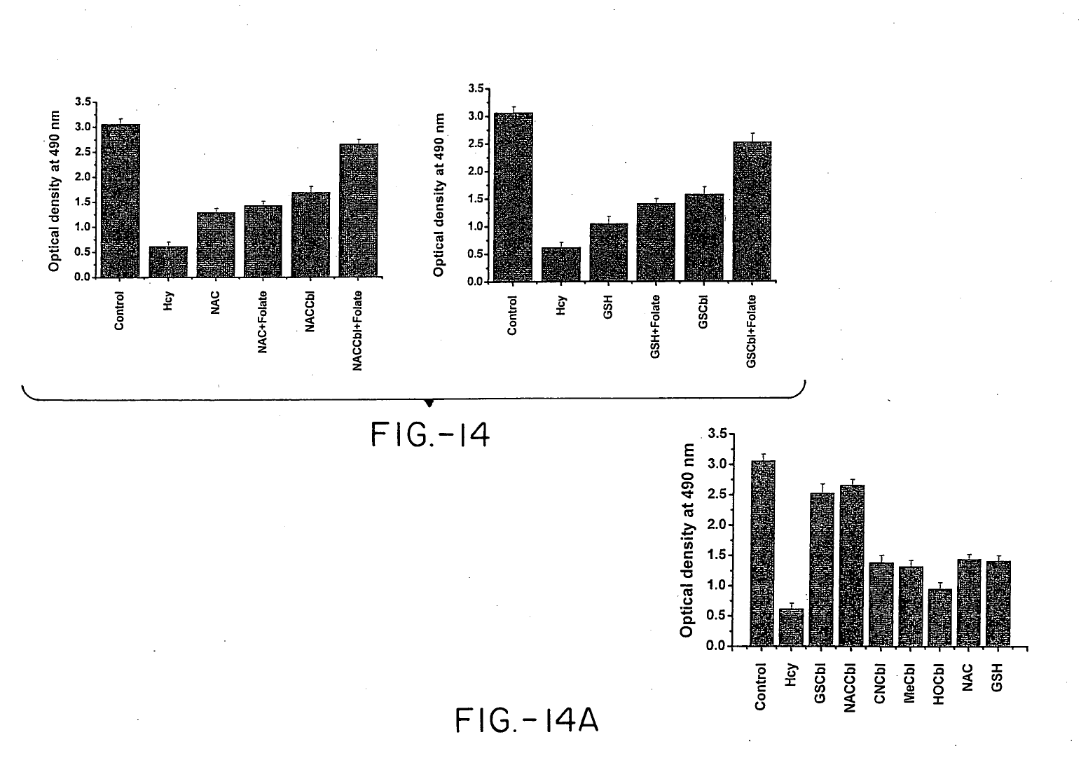

A direct comparison of the protection against homocysteine-induced damage demonstrated that NACCbl and GSCbl are superior to the free thiols. (FIGS. 14, 14(a). FIG. 14 illustrates the effect of the free thiols NAC (45 μm) and GSH (100 μM) versus NACCbl (12.5 μM) or GSCbl (15 μM) in the absence or presence of folate (25 μm) on protecting SK-HEP-1 cells from Hcy (30 μM). FIG. 14a illustrates the effects of cobalamins in the presence of folate (25 μM) on protecting T SK-HEP-1 cells from Hcy (30 μM). GSCbl=15 μM; NACCbl=12.5 μM; CNCbl=15.0 μM; MeCbl=12.5 μM; HOCbl=15.5 μM. The protective effects of NAC (45 μM) and GSH (100 μM) in the presence of folate (25 μM) are also shown for comparison purposes. The data is set forth below.

| Condition | [Hcy] (μM) | Mean | SDM |
|---|---|---|---|
| Control | 0 | 3.06117 | 0.10953 |
| Hcy | 30 | 0.616 | 0.09193 |
| NAC | 30 | 1.29633 | 0.07974 |
| NACCbl | 30 | 1.69583 | 0.11965 |
| NAC + Folate | 30 | 1.43783 | 0.08293 |
| NACCbl + Folate | 30 | 2.66117 | 0.09405 |

[NAC] = 45 μM
[Folate] = 25 μM
[NACCbl] = 12.5 μM

| Condition | [Hcy] (μM) | Mean | SDM |
|---|---|---|---|
| Control | 0 | 3.06117 | 0.10953 |
| HCy | 30 | 0.616 | 0.09193 |
| GSCbl | 30 | 2.52433 | 0.15311 |
| NACCbl | 30 | 2.66117 | 0.09405 |
| CNCbl | 30 | 1.38333 | 0.12226 |
| MeCbl | 30 | 1.32217 | 0.10209 |
| HOCbl | 30 | 0.94983 | 0.10809 |
| NAC | 30 | 1.43783 | 0.08293 |
| GSH | 30 | 1.40933 | 0.09033 |

[GSH] = 100 μM
[NAC] = 45 μM
[Folate] = 25 μM
[GSCbl] = 15 μM; [NACCbl] = 12.5 μM; [CNCbl] = 15 μM;
[MeCbl] = 12.5 μM; [HOCbl] = 17.5 μM.

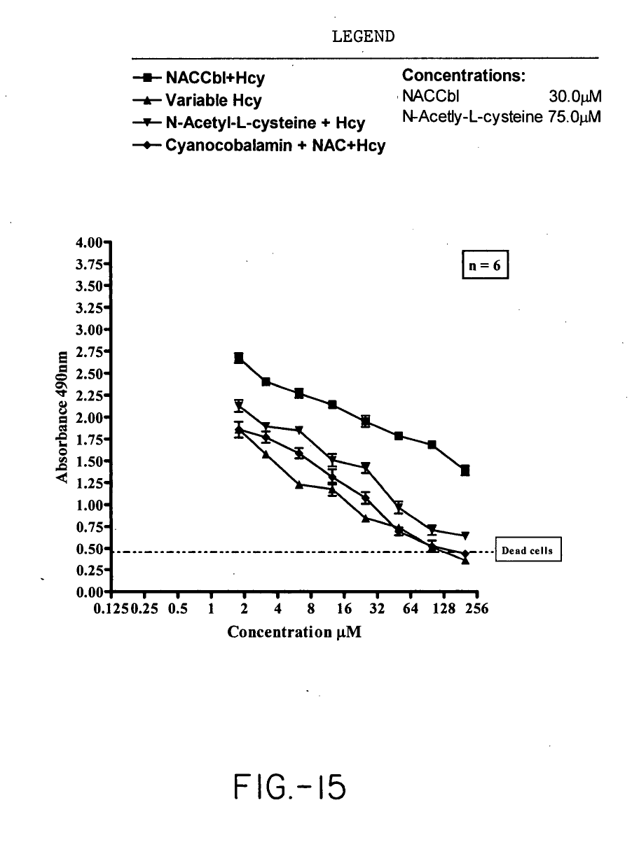

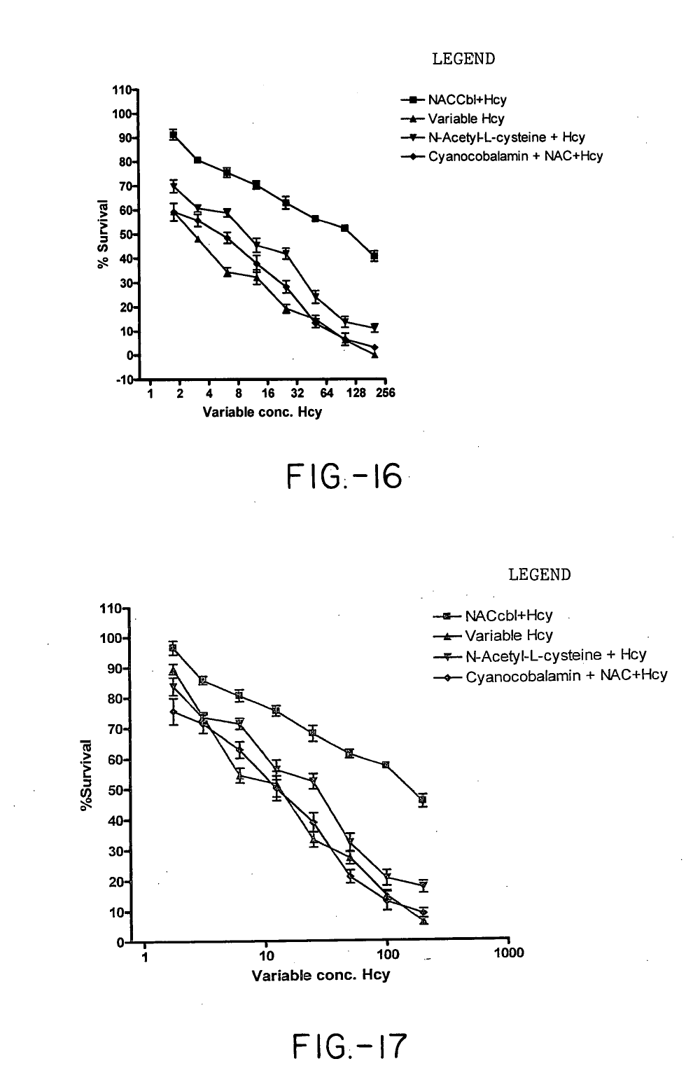

A comparison of the protective effects of NACCbl, NAC and cyanocobalamin+NAC against variable Hcy concentration is shown in FIG. 15. The results demonstrate that NACCbl (30 μM) is superior to either NAC (75 μM) alone or in combination with CNCbl (cyanocobalamin+NAC) in preventing cell death. Table 15 and Table 15 (a) below show the results for cell death as characterized by absorbance at 490 nm for NACCbl, variable Hcy, NAC and cyanocobalamin+NAC. The data was normalized (log 2 and log 10) as shown in FIGS. 16 and 17 and Tables 16 and 17, respectively, below. Normalization reflects that protection with NACCbl is superior to that of NAC or cyanocobalamin+NAC.

TABLE 15
Absorbance at 490 nm (n = 6)

| Variable | Variable Hcy | | | | | |
|---|---|---|---|---|---|---|
| Conc. of Hcy | Y1 | Y2 | Y3 | Y4 | Y5 | Y6 |
| 200.000 | 0.330 | 0.312 | 0.390 | 0.302 | 0.421 | 0.406 |
| 100.000 | 0.654 | 0.449 | 0.466 | 0.501 | 0.522 | 0.510 |
| 50.000 | 0.678 | 0.881 | 0.602 | 0.770 | 0.724 | 0.779 |
| 25.000 | 0.692 | 0.956 | 0.876 | 0.882 | 0.937 | 0.740 |
| 12.500 | 1.104 | 1.001 | 1.042 | 1.114 | 1.324 | 1.479 |
| 6.250 | 1.304 | 1.406 | 1.227 | 1.176 | 1.150 | 1.119 |
| 3.125 | 1.543 | 1.487 | 1.656 | 1.630 | 1.593 | 1.558 |
| 1.780 | 1.746 | 1.932 | 1.950 | 1.830 | 1.837 | 1.884 |
| 0.000 | 2.109 | 2.110 | 1.999 | 2.056 | 2.100 | 1.936 |
| Variable | NACCbl + Hcy | | | | | |
| Conc. of Hcy | Y1 | Y2 | Y3 | Y4 | Y5 | Y6 |
| 200.000 | 1.378 | 1.167 | 1.330 | 1.456 | 1.491 | 1.537 |
| 100.000 | 1.678 | 1.592 | 1.694 | 1.773 | 1.732 | 1.639 |
| 50.000 | 1.678 | 1.832 | 1.881 | 1.730 | 1.740 | 1.855 |
| 25.000 | 2.100 | 2.190 | 1.879 | 1.937 | 1.779 | 1.837 |
| 12.500 | 2.210 | 2.110 | 2.045 | 2.065 | 2.101 | 2.324 |
| 6.250 | 2.410 | 2.400 | 2.322 | 2.201 | 2.115 | 2.187 |
| 3.125 | 2.453 | 2.338 | 2.360 | 2.317 | 2.418 | 2.535 |
| 1.780 | 2.647 | 2.551 | 2.789 | 2.890 | 2.549 | 2.611 |
| 0.000 | 2.885 | 2.698 | 2.821 | 2.771 | 2.691 | 2.700 |
| Variable | N-Acetyl-L-cysteine + Hcy | | | | | |
| Conc. of Hcy | Y1 | Y2 | Y3 | Y4 | Y5 | Y6 |
| 200.000 | 0.567 | 0.668 | 0.623 | 0.490 | 0.721 | 0.788 |
| 100.000 | 0.789 | 0.842 | 0.743 | 0.589 | 0.490 | 0.799 |
| 50.000 | 0.890 | 0.821 | 1.034 | 1.227 | 0.799 | 1.045 |
| 25.000 | 1.345 | 1.501 | 1.632 | 1.378 | 1.226 | 1.447 |
| 12.500 | 1.675 | 1.232 | 1.339 | 1.590 | 1.602 | 1.622 |
| 6.250 | 1.678 | 1.789 | 1.897 | 1.899 | 1.933 | 1.905 |
| 3.725 | 1.890 | 2.003 | 1.784 | 1.946 | 1.958 | 1.801 |
| 1.780 | 2.225 | 1.903 | 1.947 | 2.310 | 2.187 | 2.206 |
| 0.000 | 2.592 | 2.476 | 2.437 | 2.678 | 2.336 | 2.447 |
| Variable | Cyanocobalamin + NAC + Hcy | | | | | |
| Conc. of Hcy | Y1 | Y2 | Y3 | Y4 | Y5 | Y6 |
| 200.000 | 0.345 | 0.421 | 0.456 | 0.567 | 0.378 | 0.471 |
| 100.000 | 0.789 | 0.634 | 0.456 | 0.447 | 0.336 | 0.490 |
| 50.000 | 0.890 | 0.576 | 0.678 | 0.732 | 0.602 | 0.693 |
| 25.000 | 1.345 | 0.898 | 0.936 | 1.121 | 1.044 | 1.117 |
| 12.500 | 1.675 | 1.056 | 1.210 | 1.226 | 1.402 | 1.339 |
| 6.250 | 1.678 | 1.538 | 1.336 | 1.669 | 1.722 | 1.590 |
| 3.725 | 1.890 | 1.578 | 1.773 | 1.609 | 1.793 | 1.993 |
| 1.780 | 2.225 | 1.567 | 1.875 | 1.690 | 1.884 | 1.921 |
| 0.000 | 2.592 | 2.389 | 2.449 | 2.201 | 2.108 | 2.557 |

TABLE 15(a)
Statistical Analysis (n = 6)
Absorbance at 490 nm

| | | | | | N-Acetyl-L- | | Cyanoco- | |
|---|---|---|---|---|---|---|---|---|
| | NACCbl + | | | | cysteine + | | balamin + | |
| Variable | Hcy | | Variable Hcy | | Hcy | | NAC + Hcy | |
| Conc. Hcy | Mean | SEM | Mean | SEM | Mean | SEM | Mean | SEM |
| 200.000 | 1.393 | 0.055 | 0.360 | 0.021 | 0.643 | 0.044 | 0.440 | 0.032 |
| 100.000 | 1.685 | 0.026 | 0.517 | 0.030 | 0.709 | 0.057 | 0.525 | 0.066 |
| 50.000 | 1.786 | 0.033 | 0.739 | 0.039 | 0.969 | 0.067 | 0.695 | 0.046 |
| 25.000 | 1.954 | 0.065 | 0.847 | 0.044 | 1.421 | 0.057 | 1.077 | 0.065 |
| 12.500 | 2.142 | 0.043 | 1.177 | 0.076 | 1.510 | 0.073 | 1.318 | 0.086 |
| 6.250 | 2.273 | 0.050 | 1.230 | 0.044 | 1.850 | 0.040 | 1.589 | 0.057 |
| 3.125 | 2.404 | 0.033 | 1.578 | 0.025 | 1.897 | 0.036 | 1.773 | 0.065 |
| 1.780 | 2.673 | 0.056 | 1.863 | 0.031 | 2.130 | 0.067 | 1.860 | 0.092 |
| 0.000 | 2.761 | 0.032 | 2.052 | 0.029 | 2.494 | 0.050 | 2.383 | 0.079 |

TABLE 16
Normalized Data Log 2 (n = 6)

| | | | | | N-Acetyl-L- | | Cyanocobalamin + | |
|---|---|---|---|---|---|---|---|---|
| Variable | NACCbl + Hcy | | Variable Hcy | | cysteine + Hcy | | NAC + Hcy | |
| Conc. Hcy | Mean | SEM | Mean | SEM | Mean | SEM | Mean | SEM |
| 200.000 | 40.781 | 2.159 | −0.033 | 0.832 | 11.135 | 1.727 | 3.108 | 1.261 |
| 100.000 | 52.298 | 1.042 | 6.164 | 1.170 | 13.738 | 2.233 | 6.493 | 2.595 |
| 50.000 | 56.302 | 1.307 | 14.935 | 1.538 | 24.035 | 2.637 | 13.203 | 1.803 |
| 25.000 | 62.926 | 2.573 | 19.208 | 1.730 | 41.900 | 2.249 | 28.283 | 2.585 |
| 12.500 | 70.387 | 1.704 | 32.253 | 2.986 | 45.397 | 2.896 | 37.811 | 3.412 |
| 6.250 | 75.524 | 1.973 | 34.347 | 1.739 | 58.837 | 1.578 | 48.512 | 2.265 |
| 3.125 | 80.699 | 1.323 | 48.077 | 0.992 | 60.687 | 1.432 | 55.775 | 2.575 |
| 1.780 | 91.341 | 2.227 | 59.351 | 1.212 | 69.880 | 2.655 | 59.239 | 3.625 |
| 0.000 | 94.824 | 1.284 | 66.798 | 1.145 | 84.288 | 1.965 | 79.876 | 3.120 |

TABLE 17
Normalized Data Log 10 (n = 6)

| | | | | | N-Acetyl-L- | | Cyanocobalamin + | |
|---|---|---|---|---|---|---|---|---|
| Variable | NACCbl + Hcy | | Variable Hcy | | cysteine + Hcy | | NAC + Hcy | |
| Conc. Hcy | Mean | SEM | Mean | SEM | Mean | SEM | Mean | SEM |
| 200.000 | 45.526 | 2.176 | 6.115 | 1.169 | 17.503 | 1.948 | 8.893 | 1.497 |
| 100.000 | 57.135 | 1.050 | 14.820 | 1.644 | 20.439 | 2.518 | 12.910 | 3.079 |
| 50.000 | 61.171 | 1.317 | 27.142 | 2.160 | 32.051 | 2.974 | 20.874 | 2.140 |
| 25.000 | 67.848 | 2.594 | 33.145 | 2.430 | 52.198 | 2.537 | 38.770 | 3.067 |
| 12.500 | 75.368 | 1.717 | 51.471 | 4.194 | 56.141 | 3.266 | 50.078 | 4.049 |
| 6.250 | 80.546 | 1.989 | 54.413 | 2.443 | 71.298 | 1.779 | 62.777 | 2.688 |
| 3.125 | 85.763 | 1.333 | 73.700 | 1.394 | 73.385 | 1.615 | 71.397 | 3.056 |
| 1.780 | 96.489 | 2.245 | 89.537 | 1.702 | 83.752 | 2.994 | 75.508 | 4.302 |
| 0.000 | 100.000 | 1.294 | 100.000 | 1.609 | 100.000 | 2.216 | 100.000 | 3.703 |

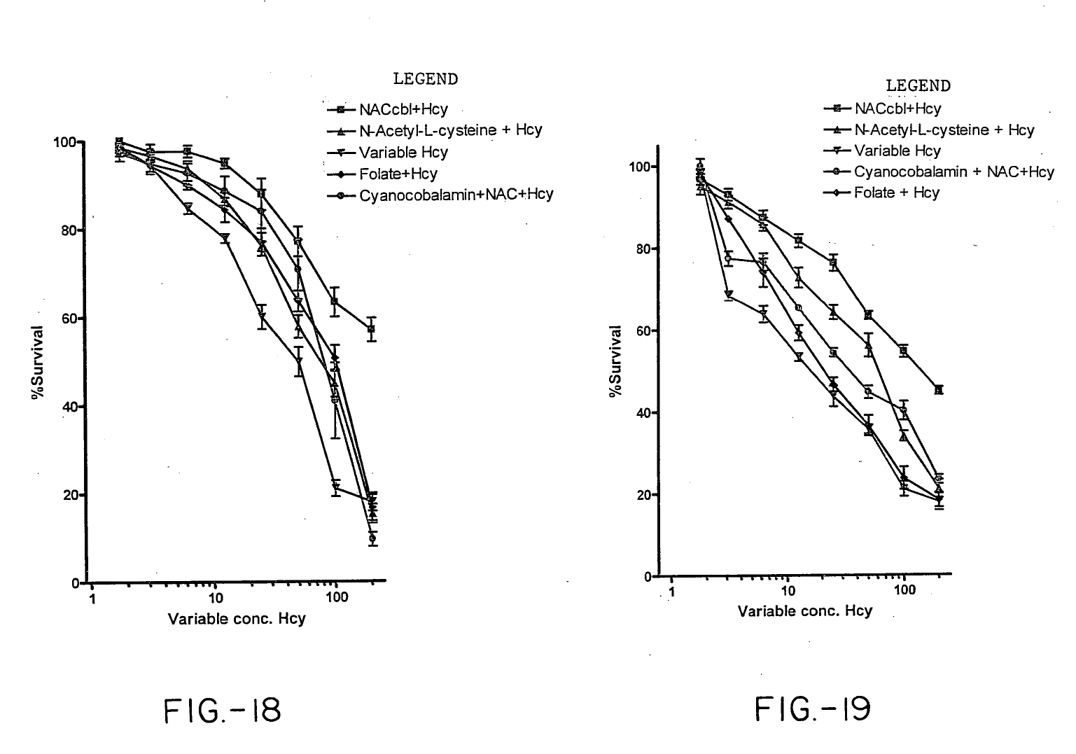

Additional cell studies were conducted using Jurkat (T-cells) and U937 (monocyte) cell lines. These experiments confirmed that other cell types are killed by homocysteine, although these cell lines are not as sensitive as the SK-HEP-1 cell line discussed in the experiments above and are thus more resistant to homocysteine. Cells were exposed to NACCbl 30 μM, NAC 75 μM, CNCbl 15 μM, and folate 30 μM. Data set forth in FIGS. 18 (U937) and 19 (Jurkat cells) showed that NACCbl is more effective at protecting these cells from death than NAC, CN Cbl or folate, especially at higher homocysteine concentrations, and that the protective effect is not just limited to SK-HEP-1 cells. In these Figures, the monocytes are N=6 and concentrations are as follows: NACCbl 30 μM, NAC 75 μM, CNCbl 15 μM, Folate 30 μM

### CONCLUSIONS

NACCbl has been shown to be stable and biologically active and to protect cells from oxidative stress damage. This novel, synthetic thiolatocobalamin was more effective than any of the other cobalamins in this activity for both homocysteine and H₂O₂-induced oxidative stress.

It will be understood by those who practice the invention and those skilled in the art that various modifications and improvements may be made to the invention without departing from the spirit of the disclosed concept. The scope of protection afforded is to be determined by the claims and by the breadth of interpretation allowed by law.

What is claimed is:

1. A method to treat, ameliorate or prevent a disease associated with or a condition of oxidative stress in an animal comprising administering an effective amount of glutathionylcobalamin.

2. The method according to claim 1 further comprising administering said glutathionylcobalamin in combination with one or more compound known to reduce serum homocysteine levels.

3. The method according to claim 2 in which said compounds known to reduce serum homocysteine levels are one or more of a folate compound and vitamin B₆.

4. The method according to claim 3 in which said compound known to reduce serum homocysteine levels is a natural isomer of reduced folate.

5. The method according to claim 4 wherein said natural isomer of reduced folate is one or more of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and their polyglutamyl derivatives.

6. The method according to claim 1 wherein said disease is selected from the group consisting of cardiovascular disease, cerebrovascular disease, peripheral vascular disease, glaucoma, Alzheimer's disease, and dementia and combinations thereof.

7. The method according to claim 1 in which the animal is a mammal.

8. The method according to claim 7 in which the animal is livestock, a domestic animal or a human.

9. A method of reducing high homocysteine levels or of reducing free radical formation levels in a human comprising administering aa effective amount of a synthetic glutathionylcobalamin.

10. A method according to claim 10 further comprising administering said glutathionylcobalamin in combination with one or more of a folate compound and vitamin B₆ in a biologically acceptable carrier.

11. A pharmaceutical composition comprising glutathionylcobalamin.

12. A pharmaceutical composition according to claim 11 wherein said glutathionylcobalamin is synthetic glutathionylcobalamin.

13. The pharmaceutical composition according to claim 12 in a biologically acceptable carrier.

14. The pharmaceutical composition according to claim 13 further comprising a folate compound.

15. The pharmaceutical composition according to claim 14 wherein the folate is a natural isomer of reduced folate.

16. The pharmaceutical composition according to claim 14 wherein the natural isomer of reduced folate is one or more of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and their polyglutamyl derivatives.

17. The pharmaceutical composition according to claim 16 further comprising vitamin B₆.

18. The pharmaceutical composition according to claim 17 further comprising folate and vitamin B₆.

19. A dietary supplement for treating an animal against diseases associated with or conditions of oxidative stress comprising glutathionylcobalamin.

20. The dietary supplement according to claim 19 wherein said glutathionylcobalamin is a synthetic glutathionylcobalamin.

21. The dietary supplement according to claim 20 which comprises one or more of human food supplements, multivitamin preparations, breakfast foods, infant formulas, complete diet and weight-loss formulas and bars; animal feed and animal feed supplements.

22. The dietary supplement according to claim 21 further comprising one of more of a folate compound and vitamin B₆.

23. A pharmaceutical composition comprising a synthetic glutathionyl-cobalamain with folate and vitamin B₆ in a biologoically acceptable carrier.

24. The pharmaceutical composition according to claim 23 wherein said folate is a reduced isomer of folate.

25. The pharmaceutical composition according to claim 24 wherein the folate is one or more of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, and their polyglutamyl derivatives.
